(19) United States
(12) Patent Application Publication
(10) Pub. No.: US 2010/0093100 A1
(43) Pub. Date: Apr. 15, 2010

(54) PROFILING METHOD USEFUL FOR CONDITION DIAGNOSIS AND MONITORING, COMPOSITION SCREENING, AND THERAPEUTIC MONITORING

(75) Inventors: Jonathan B. Chaires, Louisville, KY (US); Nichola C. Garbett, Louisville, KY (US); Albert S. Benight, Louisville, KY (US)

Correspondence Address: STITES & HARBISON, PLLC, 400 W MARKET ST, SUITE 1800, LOUISVILLE, KY, 40202-3352, US

(73) Assignees: UNIVERSITY OF LOUISVILLE RESEARCH FOUNDATION, INC., Louisville, KY (US); LOUISVILLE BIOSCIENCE, INC., Louisville, KY (US)

(21) Appl. No.: 12/561,081

(22) Filed: Sep. 16, 2009

Related U.S. Application Data
(63) Continuation-in-part of application No. 11/972,921, filed on Jan. 11, 2008.
(60) Provisional application No. 61/097,433, filed on Sep. 16, 2008.
(60) Provisional application No. 60/978,252, filed on Oct. 8, 2007.
(60) Provisional application No. 60/884,730, filed on Jan. 12, 2007.

(51) Int. Cl. G01N 33/92; G01N 33/53

(52) U.S. Cl. 436/71; 436/86; 436/94

(57) ABSTRACT

The presently-disclosed subject matter includes methods and systems for identifying biomarkers of interest, diagnosing and/or monitoring conditions of interest, assessing the efficacy of a treatment program, and composition screening. Exemplary methods include providing a sample of interest, fractionating the sample, generating thermograms, and comparing thermograms.

### RELATED APPLICATIONS

This application claims priority from U.S. Provisional Application Ser. No. 61/097,433 filed on Sep. 16, 2008, and is a continuation-in-part of commonly assigned and co-pending U.S. patent application Ser. No. 11/972,921 filed on Jan. 22, 2008. U.S. patent application Ser. No. 11/972,921 claims priority from U.S. Provisional Patent Application Ser. Nos. 60/978,252 filed Oct. 8, 2007, and 60/884,730 filed Jan. 12, 2007. The entire disclosures of U.S. Patent Application Ser. Nos. 61/097,433; 11/972,921; 60/978,252; and 60/884,730 are incorporated herein by this reference.

### GOVERNMENT INTEREST

Subject matter described herein was made with government support under Grant Number R44 CA103437 awarded by the National Cancer Institute. The United States government has certain rights in the described subject matter.

### TECHNICAL FIELD

The presently-disclosed subject matter relates to methods for condition diagnosis and monitoring, biomarker discovery, composition screening, and therapeutic monitoring using sample profiling. In particular, the presently-disclosed subject matter relates to sample fraction profiling methods that make use of fractionation techniques in combination with differential scanning calorimetery (DSC) and use of resulting thermograms. The presently-disclosed subject matter further relates to identification of sample components responsible for alteration of thermograms in subjects having a condition of interest.

### INTRODUCTION AND GENERAL CONSIDERATIONS

Biological samples (e.g., plasma, serum, blood, urine, saliva, etc.) are complex samples that contain thousands of individual polypeptides that are present in quantities that range from picograms to tens of milligrams per milliliter. The expression of specific proteins and specific changes in protein expression levels in samples from a subject can be associated with specific conditions, e.g., disease, stage or progression of a condition, infection, etc. As such, analysis of protein levels and changes in protein levels can provide information useful for purposes such as condition diagnosis and therapeutic monitoring. In the clinical setting, certain diagnostic tests include obtaining proteomic profiles of biological samples collected from a patient. Such diagnostic tests search for protein biomarkers or changes in expression of certain proteins found in biological samples, which can often be easily obtained from patients using minimally invasive, safe procedures.

A number of FDA-approved plasma and serum diagnostic assays currently exist; for example, serum and plasma electrophoresis, and a variety of immunochemical assays can be used to monitor the concentrations of specific proteins in plasma and serum. These existing low-to-moderate resolution assays have had a practical impact on medical diagnosis. Such assays can provide useful information at early stages of a disease, allowing for intervention and improved outcomes for patients, with lower associated monetary costs. However, specific protein levels or changes in protein levels associated with conditions of interest can be small, relative to the overall levels of proteins in a given fluid sample. As such, the sensitivity of a method for analyzing protein levels should be such that relatively low levels and minor fluctuations can be detected.

Developments in proteomics have brought increased interest human biological samples containing protein, such as the human plasma and serum proteome, as a source for biomarkers of human disease. Higher resolution methods like 2-D electrophoresis and mass spectrometry, coupled with often elaborate protocols for sample preparation and fractionation, have made it possible to identify apparent changes in the composition of the less abundant proteins and peptides in plasma that correlate with particular diseases. Typically no single protein emerges from such analyses as a wholly reliable biomarker, but instead changes in the patterns of panels of proteins often serve as the best diagnostic for a particular malady. These patterns often involve protein or peptide components of plasma that are present in low concentrations.

Interest in the array of existing proteins in a patient's biological sample has thus evolved to consider in more detail the low molecular weight peptides the sample, e.g., serum, which represent a mixture of small intact proteins plus degradation fragments of larger proteins. The low molecular weight region of the serum proteome has been dubbed the “peptidome,” and has been touted as a “treasure trove of diagnostic information that has largely been ignored . . . ” See Liotta and Petricoin, J. Clin. Invest. (2006), and Liotta, et al., Nature (2003). Although some consider the peptidome “unidentified flying peptides,” and have questioned the reliability of peptidome SELDI (surface-enhanced laser desorption ionization) patterns as a meaningful diagnostic until the functions of all of the peptide peaks in the peptidome have been properly identified, mass spectrometry, in particular SELDI methods, have made the peptidome accessible for analysis. See Anderson, Proteomics (2005). Many components of the “peptidome” have been found to be complexed with more abundant serum proteins, particular human serum albumin (HAS) and immunoglobulins. Such findings led to the concept of an “interactome,” which introduces the added complexity that serum and plasma can be “comprised of a ‘network’ of protein-protein and peptide-protein interactions,” in which potential biomarkers are bound to the more abundant proteins within the fluid. See Zhou, et al., Electrophoresis (2004). Interestingly, the paper that introduced the “interactome” concept concludes by saying that “the discovery of novel biomarkers in serum/plasma requires new biochemical and analytical approaches, and, most importantly, it is clear that no single sample preparation or detection method will suffice if biomarker investigations are to be broadly successful using current technologies.” See Zhou, et al., Electrophoresis, (2004).

Ten proteins make up 90% of the mass of plasma (by weight). These are, in order of abundance: albumin, IgG, Fibrinogen, Transferrin, IgA, α₂-macroglobulin, α₁-antitrypsin, complement C3, IgM and Haptoglobin. Another 12 proteins account for another 9% of the plasma mass, the 3 most abundant of which are the apolipoproteins A1 and B, and α₁-acid glycoprotein. Twenty-two proteins thus comprise 99% of the mass of plasma, making it challenging to fractionate and quantify the remaining 1%.

The FDA-approved serum protein electrophoresis method monitors changes in the most abundant protein population. See O'Connell, et al., Am. Fam. Physician (2005). However, this method has sensitivity limitations and does not adequately detect changes in less-abundant proteins. Additionally, the equipment necessary for practicing this serum protein electrophoresis method is costly to obtain and maintain.

More recently, 2-D gel electrophoresis and mass spectrometry assays have been developed, which allow for detection of the least abundant components of plasma; however, samples must be prepared by following laborious prefractionation protocols to rid the plasma/serum of the proteins present in high concentrations. See Anderson, Proteomics (2005); Anderson and Anderson, Electrophoresis (1991); Gygi and Aebersold, Curr Opin Chem Biol (2000); Liotta, et al., JAMA (2001); Yates, Trends Genet (2000); and Adkin, et al., Mol Cell Proteomics (2002). Additionally, these assays are time consuming and the equipment necessary for practicing these methods can be costly to obtain and maintain.

Although, the proteomes of biological samples, e.g., plasma proteome, holds great promise as a convenient specimen for disease diagnosis and therapeutic monitoring, existing assays and technologies have various drawbacks, including sensitivity limitations, time and efficiency limitations, and associated costs that can be prohibitive. Additionally, existing assays and technologies do not fully exploit the biological samples as a source for biomarkers. For example, electrophoresis and mass spectrometry both separate plasma proteins based on protein size and charge, but assays and technologies based on other physical properties of protein are lacking.

Accordingly, there remains a need in the art for a method for obtaining and exploiting proteomic profiles of samples, which will address the above-mentioned drawbacks of existing technologies.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes a method of identifying biomarkers useful for diagnosing a condition of interest in a subject, which includes: providing a test sample associated with the condition of interest; fractionating the test sample to obtain fractions of the test sample; generating a signature thermogram for at least one fraction of the test sample; comparing the signature thermogram to a sibling standard thermogram; and determining whether the signature thermogram is a good simulation or a poor simulation of the sibling standard thermogram.

In some embodiments, the sibling standard thermogram is a sibling positive standard thermogram generated using a positive control sample including a candidate biomarker. In some embodiments, the candidate biomarker is selected from a protein, a nucleic acid, a phospho lipid, and a small organic molecule. In some embodiments, the candidate biomarker is identified as an actual biomarker when the signature thermogram of a fraction of the test sample is a good simulation of sibling positive standard thermogram of the candidate biomarker.

In some embodiments the method of identifying useful biomarkers includes providing a negative control sample associated with an absence of the condition of interest; fractionating the control sample to obtain sibling fractions of the control sample; wherein the sibling standard thermogram is a sibling negative standard thermogram generated for a sibling fraction of the negative control sample; and identifying a fraction of the test sample having a unique component relative to the sibling fraction of the negative control sample due to the signature thermogram being a poor simulation of the sibling negative standard thermogram. In some embodiments, the method also includes testing the fraction of the test sample having a unique component to determine the identity of the unique component; and classifying the identified unique component as a biomarker useful for diagnosing the condition of interest.

In some embodiments of the method of identifying biomarkers, the fractionating is conducted using gel filtration, gel electrophoresis, chromatographic fractionation, separation columns, immunoaffinity, centrifugation, mass spectroscopy, bioinformatic fractionation, or combinations thereof. In some embodiments, the chromatographic fractionation is conducted using gel filtration chromatography, liquid chromatography (LC), LC-mass spectroscopy (LC-MS), affinity chromatography, or high pressure liquid chromatography (HPLC). In some embodiments, the mass spectroscopy is conducted using high-resolution LC-MS/MS, surface-enhanced laser desorption/ionization-time-of-flight (SELDI-TOF) MS, or matrix-assisted laser desorption/ionization-time-of-flight (MALDI-TOF) MS. In some embodiments, the fractionation results in fraction including different size classes of proteins.

In some embodiments of the method of identifying biomarkers, the condition of interest is selected from the group consisting of: a cancer, an autoimmune disease, and a microbial infection. In some embodiments, the condition is a cancer selected from the group consisting of: brain cancer, central nervous system (CNS) cancer, cervical cancer, endometrial cancer, lung cancer, leukemia, lymphoma, melanoma, multiple myeloma, ovarian cancer, and vulvar cancer. In some embodiments, the cancer is selected from: a cancer of glial cells, including astrocytes, oligodendrocytes, ependymal cells; a cancer of neurons; a cancer of lymphatic tissue; a cancer of blood vessels; a cancer of cranial nerves; a cancer of the brain envelope; a cancer of the pitutitary gland; a cancer of the pineal gland; a metastatic cancer of the brain, and a secondary cancer, wherein the primary cancer is a brain cancer. In some embodiments, the cancer is selected from: grade 1 astrocytoma, grade 2 astrocytoma, grade 3 astrocytoma, and glyoblastoma mutiforme. In some embodiments, the condition of interest is a stage of cervical cancer selected from: moderate cervical dysplasia (CIN II), early stage cervical cancer, and stage IVB cervical cancer. In some embodiments, the condition of interest is an autoimmune disease. In some embodiments, the autoimmune disease is selected from: rheumatoid arthritis, multiple sclerosis, and systemic lupus. In some embodiments, the condition of interest is caused by a bacterial infection. In some embodiments, the condition is Lyme disease. In some embodiments, the condition of interest is caused by a viral infection. In some embodiments, the condition is selected from: Dengue fever, and hepatitis. In some embodiments, the condition of interest is selected from: amyotrophic lateral sclerosis (ALS), anemia, cardiac disease, diabetes, and renal disease.

In some embodiments of the method of identifying biomarkers, the samples are selected from: plasma sample, serum sample, a blood sample, an ascites fluid sample, a cerebral spinal fluid sample, a peritoneal fluid sample, a saliva sample, a senovial fluid sample, an ocular fluid sample, and a urine sample.

The presently-disclosed subject matter includes a system for identifying biomarkers useful for diagnosing a condition of interest in a subject, comprising: means for accepting a sample; means for fractionating a test sample and/or a control to obtain fractions of the test sample and/or the control sample; means for generating a signature thermogram for at least one fraction of the test sample and/or a standard thermogram for at least one fraction of the control sample; means for comparing a signature thermogram to a sibling standard thermogram; and means for determining whether the signature thermogram is a good simulation or a poor simulation of the sibling standard thermogram. In some embodiments, the system also includes means for testing the fraction of the test sample having a unique component to determine the identity of the unique component, which unique component is identified as a biomarker useful for diagnosing the condition of interest.

The presently-disclosed subject matter includes a method of diagnosing or monitoring a condition of interest in a subject, which includes providing a test sample to a subject; fractionating the test sample to obtain fractions of the test sample; generating a signature thermogram for each fraction of the test sample; comparing a signature thermogram with a sibling standard thermogram and/or a sibling signature thermogram; and identifying a status of the subject. In some embodiments, the standard thermogram is selected from a positive standard thermogram associated with a presence of the condition of interest, and a negative standard thermogram associated with an absence of the condition of interest. In some embodiments, the method also includes providing multiple standard thermograms associated with different conditions of interest. In some embodiments, the multiple positive standard thermograms include positive standard thermograms for different stages of a condition of interest.

In some embodiments of the method of diagnosing or monitoring a condition of interest in a subject, the method also includes identifying the status of the subject as having the condition of interest when the signature thermogram of a fraction of the test sample is a poor simulation of the negative standard thermogram; and/or the signature thermogram of a fraction of the test sample is a good simulation of the positive standard thermogram; and identifying the status of the subject as lacking the condition of interest when the signature thermogram of a fraction of the test sample is a good simulation of the negative standard thermogram; and/or the signature thermogram of a fraction of the test sample is a poor simulation of the positive standard thermogram.

In some embodiments of the method of diagnosing or monitoring a condition of interest in a subject, the method also includes providing a second test sample obtained from the subject at a time point that is differs from a time point that the test sample is obtained; fractionating the second test sample to obtain fractions of the second test sample; generating a signature thermogram for a fraction of the second test sample; comparing the signature thermogram of the second test sample to the sibling signature thermogram of the test sample; and identifying the status of the subject has having changed if the signature thermogram of the second test sample is a poor simulation of the sibling standard thermogram of the test sample.

In some embodiments of the method of diagnosing or monitoring a condition of interest in a subject, the method also includes providing a control sample; fractionating the control sample to obtain sibiling fractions of the control sample; generating a sibling standard thermogram for the sibling fractions of the control sample; comparing the signature thermogram with the sibling standard thermogram. In some embodiments, the control sample is selected from: a positive control sample, wherein a series of sibling positive standard thermograms are generated; and a negative control sample, wherein a series of sibling negative standard thermograms are generated. In some embodiments, the method also includes identifying the status of the subject as having the condition of interest when at least one signature thermogram of a fraction of the test sample is a poor simulation of the sibling negative standard thermogram; and/or at least one signature thermogram of a fraction of the test sample is a good simulation of the sibling positive standard thermogram; and identifying the status of the subject as lacking the condition of interest when each signature thermogram of the fractions of the test sample is a good simulation of each sibling negative standard thermogram; and/or each signature thermogram of the fractions of the test sample is a poor simulation of each sibling positive standard thermogram.

In some embodiments of the method of diagnosing or monitoring a condition of interest, the fractionating is conducted using gel filtration, gel electrophoresis, chromatographic fractionation, separation columns, immunoaffinity, centrifugation, mass spectroscopy, bioinformatic fractionation, or combinations thereof. In some embodiments, the chromatographic fractionation is conducted using gel filtration chromatography, liquid chromatography (LC), LC-mass spectroscopy (LC-MS), affinity chromatography, or high pressure liquid chromatography (HPLC). In some embodiments, the mass spectroscopy is conducted using high-resolution LC-MS/MS, surface-enhanced laser desorption/ionization-time-of-flight (SELDI-TOF) MS, or matrix-assisted laser desorption/ionization-time-of-flight (MALDI-TOF) MS. In some embodiments, the fractionation results in fraction including different size classes of proteins.

In some embodiments of the method of diagnosing or monitoring a condition of interest, the condition of interest is selected from the group consisting of: a cancer, an autoimmune disease, and a microbial infection. In some embodiments, the condition is a cancer selected from the group consisting of: brain cancer, central nervous system (CNS) cancer, cervical cancer, endometrial cancer, lung cancer, leukemia, lymphoma, melanoma, multiple myeloma, ovarian cancer, and vulvar cancer. In some embodiments, the cancer is selected from: a cancer of glial cells, including astrocytes, oligodendrocytes, ependymal cells; a cancer of neurons; a cancer of lymphatic tissue; a cancer of blood vessels; a cancer of cranial nerves; a cancer of the brain envelope; a cancer of the pitutitary gland; a cancer of the pineal gland; a metastatic cancer of the brain, and a secondary cancer, wherein the primary cancer is a brain cancer. In some embodiments, the cancer is selected from: grade 1 astrocytoma, grade 2 astrocytoma, grade 3 astrocytoma, and glyoblastoma mutiforme. In some embodiments, the condition of interest is a stage of cervical cancer selected from: moderate cervical dysplasia (CIN II), early stage cervical cancer, and stage IVB cervical cancer. In some embodiments, the condition of interest is an autoimmune disease. In some embodiments, the autoimmune disease is selected from: rheumatoid arthritis, multiple sclerosis, and systemic lupus. In some embodiments, the condition of interest is caused by a bacterial infection. In some embodiments, the condition is Lyme disease. In some embodiments, the condition of interest is caused by a viral infection. In some embodiments, the condition is selected from: Dengue fever, and hepatitis. In some embodiments, the condition of interest is selected from: amyotrophic lateral sclerosis (ALS), anemia, cardiac disease, diabetes, and renal disease.

In some embodiments of the method of diagnosing or monitoring a condition of interest, the samples are selected from: plasma sample, serum sample, a blood sample, an ascites fluid sample, a cerebral spinal fluid sample, a peritoneal fluid sample, a saliva sample, a senovial fluid sample, an ocular fluid sample, and a urine sample.

The presently-disclosed subject matter includes a system for diagnosing or monitoring a condition of interest in a subject, which includes means for accepting a sample; means for fractionating a test sample and/or a control to obtain fractions of the test sample and/or the control sample; means for generating a signature thermogram for at least one fraction of the test sample and/or a standard thermogram for at least one fraction of the control sample; means for comparing a signature thermogram with a sibling standard thermogram and/or a sibling signature thermogram; and means for determining whether the signature thermogram is a good simulation or a poor simulation of the sibling standard thermogram and/or a sibling signature thermogram.

The presently-disclosed subject matter includes a method of assessing a treatment program for a subject, comprising: providing a first test sample obtained from the subject at a first time point of interest; fractionating the first test sample to obtain a first series of fractions; generating a first series of signature thermograms for the first series of fractions of the first test sample; providing a second test sample obtained from the subject at a second time point of interest; fractionating the second test sample to obtain a second series of fractions; generating a second series of signature thermograms for the second series of fractions of the second test sample; comparing the first series of signature thermograms to the second series of signature thermograms; and identifying the presence or absence of a change in the condition of interest. In some embodiments, the first time point of interest occurs before the initiation of the treatment program, and the second time point of interest occurs after the initiation of the treatment program.

In some embodiments, the method of assessing a treatment program for a subject also includes identifying the treatment program as maintaining the status of the subject when each second signature thermogram of the fractions of the second sample is a good simulation of the sibling first signature thermograms of the sibling fractions of the first sample; and identifying the treatment program as changing the status of the subject when at least one second signature thermogram of the fractions of the second sample is a poor simulation of the sibling first signature thermogram of the sibling fraction of the first sample.

In some embodiments of the method of assessing a treatment program for a subject, the fractionating is conducted using gel filtration, gel electrophoresis, chromatographic fractionation, separation columns, immunoaffinity, centrifugation, mass spectroscopy, bioinformatic fractionation, or combinations thereof. In some embodiments, the chromatographic fractionation is conducted using gel filtration chromatography, liquid chromatography (LC), LC-mass spectroscopy (LC-MS), affinity chromatography, or high pressure liquid chromatography (HPLC). In some embodiments, the mass spectroscopy is conducted using high-resolution LC-MS/MS, surface-enhanced laser desorption/ionization-time-of-flight (SELDI-TOF) MS, or matrix-assisted laser desorption/ionization-time-of-flight (MALDI-TOF) MS. In some embodiments, the fractionation results in fraction including different size classes of proteins.

In some embodiments of the method of assessing a treatment program for a subject, the condition of interest is selected from the group consisting of: a cancer, an autoimmune disease, and a microbial infection. In some embodiments, the condition is a cancer selected from the group consisting of: brain cancer, central nervous system (CNS) cancer, cervical cancer, endometrial cancer, lung cancer, leukemia, lymphoma, melanoma, multiple myeloma, ovarian cancer, and vulvar cancer. In some embodiments, the cancer is selected from: a cancer of glial cells, including astrocytes, oligodendrocytes, ependymal cells; a cancer of neurons; a cancer of lymphatic tissue; a cancer of blood vessels; a cancer of cranial nerves; a cancer of the brain envelope; a cancer of the pitutitary gland; a cancer of the pineal gland; a metastatic cancer of the brain, and a secondary cancer, wherein the primary cancer is a brain cancer. In some embodiments, the cancer is selected from: grade 1 astrocytoma, grade 2 astrocytoma, grade 3 astrocytoma, and glyoblastoma mutiforme. In some embodiments, the condition of interest is a stage of cervical cancer selected from: moderate cervical dysplasia (CIN II), early stage cervical cancer, and stage IVB cervical cancer. In some embodiments, the condition of interest is an autoimmune disease. In some embodiments, the autoimmune disease is selected from: rheumatoid arthritis, multiple sclerosis, and systemic lupus. In some embodiments, the condition of interest is caused by a bacterial infection. In some embodiments, the condition is Lyme disease. In some embodiments, the condition of interest is caused by a viral infection. In some embodiments, the condition is selected from: Dengue fever, and hepatitis. In some embodiments, the condition of interest is selected from: amyotrophic lateral sclerosis (ALS), anemia, cardiac disease, diabetes, and renal disease.

In some embodiments of the method of assessing a treatment program for a subject, the samples are selected from: plasma sample, serum sample, a blood sample, an ascites fluid sample, a cerebral spinal fluid sample, a peritoneal fluid sample, a saliva sample, a senovial fluid sample, an ocular fluid sample, and a urine sample.

The presently-disclosed subject matter includes a method of screening for a composition useful for treating a condition of interest, which includes interacting a sample associated with the condition of interest with a candidate composition; fractionating the sample to obtain a series of fractions; generating a series of signature thermograms for the series of fractions; comparing the series of signature thermograms to sibling standard thermograms; and determining the utility of the candidate composition.

In some embodiments of the method of screening for a composition useful for treating a condition of interest, the fractionating is conducted using gel filtration, gel electrophoresis, chromatographic fractionation, separation columns, immunoaffinity, centrifugation, mass spectroscopy, bioinformatic fractionation, or combinations thereof. In some embodiments, the chromatographic fractionation is conducted using gel filtration chromatography, liquid chromatography (LC), LC-mass spectroscopy (LC-MS), affinity chromatography, or high pressure liquid chromatography (HPLC). In some embodiments, the mass spectroscopy is conducted using high-resolution LC-MS/MS, surface-enhanced laser desorption/ionization-time-of-flight (SELDI-TOF) MS, or matrix-assisted laser desorption/ionization-time-of-flight (MALDI-TOF) MS. In some embodiments, the fractionation results in fraction including different size classes of proteins.

In some embodiments of the method of screening for a composition useful for treating a condition of interest, the condition of interest is selected from the group consisting of: a cancer, an autoimmune disease, and a microbial infection. In some embodiments, the condition is a cancer selected from the group consisting of: brain cancer, central nervous system (CNS) cancer, cervical cancer, endometrial cancer, lung cancer, leukemia, lymphoma, melanoma, multiple myeloma, ovarian cancer, and vulvar cancer. In some embodiments, the cancer is selected from: a cancer of glial cells, including astrocytes, oligodendrocytes, ependymal cells; a cancer of neurons; a cancer of lymphatic tissue; a cancer of blood vessels; a cancer of cranial nerves; a cancer of the brain envelope; a cancer of the pitutitary gland; a cancer of the pineal gland; a metastatic cancer of the brain, and a secondary cancer, wherein the primary cancer is a brain cancer. In some embodiments, the cancer is selected from: grade 1 astrocytoma, grade 2 astrocytoma, grade 3 astrocytoma, and glyoblastoma mutiforme. In some embodiments, the condition of interest is a stage of cervical cancer selected from: moderate cervical dysplasia (CIN II), early stage cervical cancer, and stage IVB cervical cancer. In some embodiments, the condition of interest is an autoimmune disease. In some embodiments, the autoimmune disease is selected from: rheumatoid arthritis, multiple sclerosis, and systemic lupus. In some embodiments, the condition of interest is caused by a bacterial infection. In some embodiments, the condition is Lyme disease. In some embodiments, the condition of interest is caused by a viral infection. In some embodiments, the condition is selected from: Dengue fever, and hepatitis. In some embodiments, the condition of interest is selected from: amyotrophic lateral sclerosis (ALS), anemia, cardiac disease, diabetes, and renal disease.

In some embodiments of the method of screening for a composition useful for treating a condition of interest, the samples are selected from: plasma sample, serum sample, a blood sample, an ascites fluid sample, a cerebral spinal fluid sample, a peritoneal fluid sample, a saliva sample, a senovial fluid sample, an ocular fluid sample, and a urine sample. The presently-disclosed subject matter includes a system for screening for a composition useful for treating a condition of interest, which includes means for accepting a sample; means for fractionating the sample to obtain a series of fractions; means for generating a series of signature thermograms for the series of fractions; and means for comparing the series of signature thermograms to sibling standard thermograms.

The presently-disclosed subject matter includes a method of screening a composition for plasma protein interactions, comprising: interacting the composition with a first plasma sample; fractionating the first plasma sample to obtain a first series of fractions; generating a first series of signature thermograms for the first series of fractions; comparing the first series of signature thermograms to sibling negative standard thermograms associated with an absence of plasma protein interactions; and/or a sibling second series of signature thermogram generated using a second series of fractions from a second plasma sample not interacted with the composition; and identifying the composition as lacking substantial plasma protein interactions when the first series of signature thermograms are good simulations of the sibling negative standard thermograms, and/or the sibling second series of signature thermograms.

In some embodiments of the method of screening a composition for plasma protein interactions, the fractionating is conducted using gel filtration, gel electrophoresis, chromatographic fractionation, separation columns, immunoaffinity, centrifugation, mass spectroscopy, bioinformatic fractionation, or combinations thereof. In some embodiments, the chromatographic fractionation is conducted using gel filtration chromatography, liquid chromatography (LC), LC-mass spectroscopy (LC-MS), affinity chromatography, or high pressure liquid chromatography (HPLC). In some embodiments, the mass spectroscopy is conducted using high-resolution LC-MS/MS, surface-enhanced laser desorption/ionization-time-of-flight (SELDI-TOF) MS, or matrix-assisted laser desorption/ionization-time-of-flight (MALDI-TOF) MS. In some embodiments, the fractionation results in fraction including different size classes of proteins.

The presently-disclosed subject matter includes a system for screening a composition for plasma protein interactions, which includes means for fractionating a plasma sample to obtain a first series of fractions; means for generating a first series of signature thermograms for the first series of fractions; means for comparing the first series of signature thermograms to sibling negative standard thermograms associated with an absence of plasma protein interactions; and/or a sibling second series of signature thermogram generated using a second series of fractions from a second plasma sample not interacted with the composition; and means for identifying the composition as lacking substantial plasma protein interactions when the first series of signature thermograms are good simulations of the sibling negative standard thermograms, and/or the sibling second series of signature thermograms.

### BRIEF DESCRIPTION OF THE DRAWINGS

FIG. 1 is a schematic representation of an exemplary differential scanning calorimeter (DSC), depicting sample (S) and reference (R) cells that are kept in thermal balance by heaters controlled by feedback electronics as both cells are heated at a precisely controlled rate (ΔT₂);

FIG. 2 includes an exemplary thermogram for a two-state denaturation of a protein;

FIG. 3 includes thermograms obtained by DSC, including thermograms for various individual proteins, as well as a thermogram of the weighted sum of the group of individual proteins (solid lines representing 16 individual proteins, and the dashed line for the sum); the individual proteins were weighted according to their actual known concentration in plasma, and the individual proteins were then summed to yield the dashed line, which compares well with actual experimental thermograms of plasma from healthy individuals as shown in FIG. 6A;

FIG. 4A includes two superimposed thermograms for “normal” subjects and for subjects suffering from Lyme disease;

FIG. 4B includes the quantile plots obtained after integrating and normalizing the thermograms of FIG. 4A;

FIG. 4C includes the quantile-quantile plot obtained by plotting the normal quantile (x-axis) of FIG. 4B against the Lyme quantile (y-axis) of FIG. 4B;

FIG. 5 includes an elution profile (left panel) of a sample fractionated using gel filtration chromatography, and a series of thermograms (right panel) for each of multiple fractions of the sample;

FIG. 6A includes an average thermogram of plasma calculated from samples obtained from 15 normal subjects, where the average thermogram is the black solid line, the standard deviation at each temperature is indicated by the gray shading, and where the vertical dashed line is the first moment of the thermogram;

FIG. 6B includes an average thermogram generated using plasma samples obtained from 100 normal subjects;

FIG. 6C includes an average thermogram generated using plasma samples obtained from normal subjects, and an average thermogram generated using cerebral spinal fluid (CSF) obtained from normal subjects;

FIG. 7 includes a series of thermograms for the denaturation of individual purified plasma proteins, including α₁-antitrypsin, transferrin, α₁-acid glycoprotein, complement C3, c-reactive protein, haptoglobin, prealbumin, α₂-macroglobulin, complement C4, α₁-antichymotrypsin, IgM, albumin, IgG, fibrinogen, IgA, and ceruloplasmin;

FIG. 8 includes Panel A, showing a series of thermograms (solid lines) for the 16 most abundant plasma proteins, and a calculated thermogram (dashed line) obtained from the sum of the weighted contributions of the 16 most abundant plasma proteins; and Panel B, showing thermograms obtained from mixtures of pure plasma proteins mixed at concentrations that mimic their known average concentrations in normal plasma, where the gray curve is a mixture of HSA, IgG, fibrinogen, and transferrin, and the black curve is a mixture of the 16 most abundant plasma proteins;

FIG. 9 includes thermograms for samples in which albumin was removed from serum, where Panel A shows an expected thermogram (dashed line) based on the weighted sum of the most abundant proteins (solid lines) less HSA and fibrinogen, and where Panel B shows the observed experimental thermogram for albumin-depleted serum, from which HSA was removed by affinity chromatography using a SwellGel Blue™ albumin removal kit;

FIG. 10 includes a series of thermograms, where each panel compares normal plasma with plasma associated with a condition of interest; in Panel A the condition is systemic lupus; in Panel B the condition is Lyme disease; and in Panel C the condition is Rheumatoid arthritis;

FIG. 11 is a bar graph showing the relative concentrations of the major plasma proteins for normal and diseased plasma samples, where concentrations of the individual proteins were normalized with respect to the total protein concentration;

FIG. 12 includes a series of densitometric scans from stained gels for normal samples and samples associated with Rheumatoid arthritis, Lyme disease, and Lupus;

FIG. 13 is a thermogram showing the effect of added bromocresol green on a plasma thermogram;

FIG. 14 includes Panel A, having a series a plots showing the differences between an average normal thermogram, and condition of interest thermograms, including Lupus (gray), Lyme disease (black), arthritis (thick black); and Panel B showing the difference between an average normal thermogram, and a thermogram generated using a normal plasma sample to which bromocresol green was added to a final concentration of 686 μM;

FIG. 15 includes Panel A, having plasma thermograms for a normal sample (gray), and samples to which bromocresol green was added to final concentrations of 30 μM (dashed), 148 μM (thick black), 290 μM (black) or 686 μM (circles); Panel C, having plots showing the differences in the thermograms of Panel A; Panel B, having thermograms for an HSA sample (gray), and an HSA sample to which bromocresol green was added to a final concentration of 459 μM (thick black); and Panel D, having a plot showing the differences in the thermograms of Panel B;

FIG. 16 includes a series of thermograms of samples from subjects with different stages of cervical cancer, where the top panel includes a black trace showing normal plasma, a gray trace showing a sample from a patient diagnosed with moderate cervical dysplasia (CIN II), and a dashed black trace showing a sample of plasma from a diagnosed cervical cancer patient, and where the bottom panel includes a single trace showing a thermogram for plasma from a Stage IVB cervical cancer patient;

FIG. 17 includes results from serum plasma electrophoresis of the samples used to obtain the data in FIG. 16, where the plasma protein fibrinogen is indicated by the asterisk, and where only subtle differences are evident between the panels and the most pronounced change is the relative increase in the globulin region of the electrophoresis pattern seen for the stage IVB sample (arrow);

FIG. 18 includes a series of thermograms generated using plasma samples obtained from different subjects, where the top panel includes thermograms generated using samples from four normal subjects, where the middle panel includes thermograms generated using samples from four subjects diagnosed with moderate cervical dysplasia (CIN II), where the bottom panel includes thermograms generated using samples from four subjects diagnosed with cervical cancer;

FIG. 19 includes thermograms for normal subjects, and subjects diagnosed with ovarian cancer, endometrial cancer, and uterine cancer;

FIG. 20 includes thermograms for subjects with melanoma;

FIG. 21 includes thermograms of plasma obtained prospectively from diabetic subjects exhibiting subsequent differences in future kidney function, and normal subjects exhibiting good kidney function (Panel A), and a quantile-quantile plot, prepared using the thermograms of Panel A (Panel B).

FIG. 22 includes thermograms of diabetic subjects with either minimal (CAD−) or severe (CAD+) coronary artery disease, and normal subjects.

FIG. 23 includes thermograms of subjects with amyotrophic lateral sclerosis (ALS), and normal subjects.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, definitions are set forth herein to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

The presently-disclosed subject matter includes a method of diagnosing a condition of interest in a subject; a method of monitoring a condition of interest in a subject; a method for assessing the efficacy of a treatment program for a subject; a method of screening for compositions useful for treating a condition of interest; a method of identifying biomarkers for diagnosing a condition of interest in a subject; and a method of screening a composition for plasma protein interactions, including tendency of the composition to bind serum albumin.

As used herein, the term condition of interest refers to a variety of conditions, including cancers, autoimmune diseases, and microbial infections.

In some embodiments, the condition of interest can be a cancer. The term “cancer” refers to all types of cancer or neoplasm or malignant tumors found in animals, including leukemias, carcinomas, melanoma, and sarcomas. Examples of cancers include brain cancer, including, cancer of astrocytes, oligodendrocytes, ependymal cells; a cancer of neurons; a cancer of lymphatic tissue; a cancer of blood vessels; a cancer of cranial nerves; a cancer of the brain envelope; a cancer of the pitutitary gland; CNS lymphoma, CNS leukemia, metastatic cancer found in the brain; and secondary cancers wherein the primary cancer is brain cancer. Additional examples of cancers include cancer of the bladder, breast, cervix, colon, central nervous system (CNS), endometrium, head and neck, kidney, lung, non-small cell lung, leukemia, lymphoma, melanoma, multiple myeloma, mesothelioma, ovary, prostate, sarcoma, stomach, uterus, vulva, and Medulloblastoma.

By “leukemia” is meant broadly progressive, malignant diseases of the blood-forming organs and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, and carcinoma villosum.

The term “sarcoma” generally refers to a tumor which is made up of a substance like the embryonic connective tissue and is generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphomas (e.g., Non-Hodgkin Lymphoma), immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

Additional cancers include, for example, Hodgkin's Disease, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, thyroid cancer, neuroblastoma, esophageal cancer, genitourinary tract cancer, malignant hypercalcemia, cervical cancer, endometrial cancer, and adrenal cortical cancer.

In some embodiments, the condition of interest can be an autoimmune disease. The term “autoimmune disease” refers to all types of conditions arising from an abnormal or excessive immune response in an animal to normal and/or non-foreign compounds, cells, or tissues in the body of the animal. Examples of autoimmune diseases include, but are not limited to, Dermatomyositis, Polymyositis, Pernicious anaemia, Primary biliary cirrhosis, Wegener's granulomatosis, Acute disseminated encephalomyelitis (ADEM), Addison's disease, Alopecia greata, Antiphospholipid antibody syndrome (APS), Autoimmune hepatitis, Crohns Disease, Diabetes mellitus type 1, Goodpasture's syndrome, Graves' disease, Narcolepsy, Guillain-Barré syndrome (GBS), Hashimoto's disease, Idiopathic thrombocytopenic purpura, Systemic lupus erythematosus, Mixed Connective Tissue Disease, Multiple sclerosis (MS), Myasthenia gravis, Pemphigus vulgaris, Rheumatoid arthritis, Sjögren's syndrome, Temporal arteritis, Ulcerative colitis, Autoimmune hemolytic anemia, Bullous pemphigoid, Vasculitis, Behçet's disease, and Coeliac disease.

In some embodiments, the condition of interest can be caused by an infection, such as a bacterial or a viral infection; such conditions include but are not limited to Lyme disease, Dengue fever, and hepatitis.

In some embodiments, the condition of interest can be another condition, including but not limited to amyotrophic lateral sclerosis (ALS), also known as Lou Gehrig's disease, anemia, cardiac disease, diabetes, renal disease, or plasma cell dyscrasias and related disorders.

In some embodiments, the condition of interest can be a particular stage of a condition, for example, a particular stage of cervical cancer, such as moderate cervical dysplasia (CIN II), early stage cervical cancer, or stage IVB cervical cancer. For another example, a particular stage of brain cancer, such as grade 1 astrocytoma, grade 2 astrocytoma, grade 3 astrocytoma, and grade 4 astrocytoma (grade 3 and/or grade 4 astrocytoma are sometimes referred to as glyoblastoma mutiforme).

As used herein, the term “subject” refers to both human and animal subjects. Thus, veterinary therapeutic uses are provided in accordance with the presently-disclosed subject matter. As such, the presently-disclosed subject matter provides for the treatment of mammals such as humans, as well as those mammals of importance due to being endangered, such as Siberian tigers; of economic importance, such as animals raised on farms for consumption by humans or animals used for scientific research, such as rabbits, rats, and mice; and/or animals of social importance to humans, such as animals kept as pets or in zoos. Examples of such animals include but are not limited to: carnivores such as cats and dogs; swine, including pigs, hogs, and wild boars; rodents such as guinea pigs and hamsters; primates such as monkeys; arthropods including insects, arachnids and crustaceans; fish; mollusks; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The methods of the presently-disclosed subject matter make use of a unique calorimetric process for obtaining profiles of components of samples (e.g., proteins, nucleic acids, phospholipids, small molecules, and other components), including fractionated samples.

Although some examples provided herein are related to the use of plasma samples, and protein components of samples, the same techniques and procedures can be applied to the analysis of other sample types and for examination of other sample components, such as nucleic acids, phospholipids, small organic molecules, etc. and their interactions.

Calorimetry provides a direct means for detecting what is perhaps the most fundamental property of chemical and biochemical reactions—heat changes. Biological calorimetry dates from the time of Lavoisier (1743-1794), who invented a calorimetric method for measuring the heats of metabolism of living animals. The presently-disclosed subject matter can make use of the high sensitivity of modern microcalorimeters, which can reliably measure heat changes of about 0.1 microcalories.

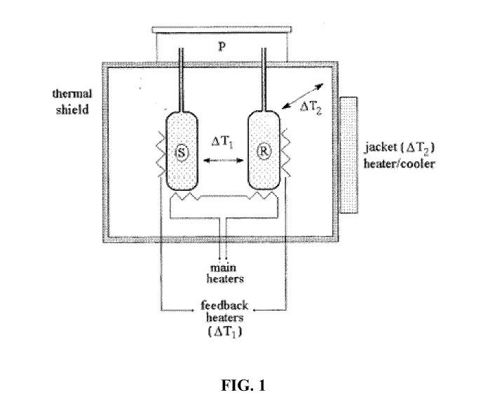

With reference to FIG. 1, an exemplary calorimeter that can be used in accordance with the presently-disclosed subject matter is a differential scanning calorimeter (DSC). In a typical DSC experiment, an aqueous solution of protein at a concentration of about 1 mg/mL or less is heated at a constant rate in a sample calorimeter cell (S) alongside an identical reference cell (R) that contains only the solvent (buffer). The electronics of the calorimeter are designed to maintain an exact thermal balance between the sample and references cells. Any chemical process in the sample cell that absorbs or releases heat results in a thermal imbalance with the reference cell, which is compensated for by a feedback heater attached to the calorimetric cells. The electrical power required to maintain the exact thermal balance of the cells is directly proportional to the apparent heat capacity of the solutions, and any change in the heat capacity is directly related to the energetics of the thermally-induced reactions that occur within the sample cell.

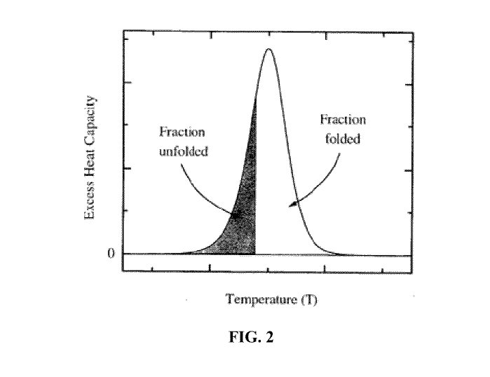

Differential scanning calorimetry (DSC) can be used for thermodynamic studies of protein denaturation. The thermodynamics of thermal-induced unfolding of proteins can be measured as directly as possible by DSC. With reference to FIG. 2, a thermogram can be obtained by DSC for a protein denaturation reaction, which expresses the excess heat capacity as a function of temperature. The area under such a thermogram is, unambiguously and directly, the enthalpy of the unfolding reaction. Integration of such a thermogram yields a transition curve (“melting curve”) from which the fractions of folded and unfolded protein forms can be calculated. The enthalpy obtained from the area of thermograms is independent of any model for the denaturation reaction that occurs in the sample cell. Such a calorimetric enthalpy provides a valuable alternative to enthalpy values obtained by use of the model-dependent van't Hoff equation (ΔH=−(δ1 nK/δT⁻¹) employing other methods, since no detailed reaction mechanism needs to be assumed. In other words, the calorimetric thermogram depends only on the initial and final states of the chemical system, and does not depend upon the manner in which the system passes from one state to the other.

Every protein has, under a given set of buffer conditions, a characteristic denaturation thermogram that is unique, and which provides a fundamental thermodynamic signature for that protein. Thermograms can be more complex than the simple two-state melting shown in FIG. 2. For more structurally complex proteins, individual structural domains within the tertiary structure can melt independently, leading to thermograms with correspondingly more complex shapes with multiple “peaks.”

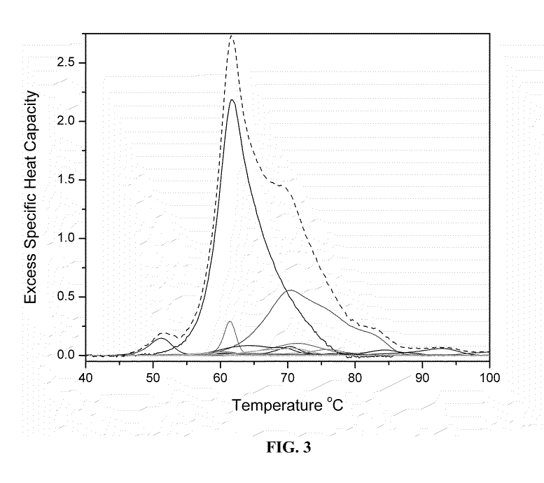

A primary DSC thermogram is an extensive property of a protein solution, and as such it is directly proportional to the mass of the protein in solution. If the weight concentration of the protein is doubled, for example, the calorimetric heat response will double. Similarly, in a solution of mixtures of proteins, the heat response will be proportional to the mass of each protein component in the mixture. Mixtures of proteins can be resolved with respect to the fundamental characteristic melting curves of their component proteins. Each protein in a noninteracting mixture will denature at its characteristic melting temperature (Tₘ) and with its characteristic melting enthalpy. The observed overall thermogram will be the weighted sum of all of the individual protein thermograms, weighted according to the mass of each component. For example, FIG. 3 contains thermograms for various individual proteins (solid lines), as well as a thermogram of the weighted sum of the group of individual proteins (dashed line).

Although some examples provided herein are related to the use of plasma samples, and protein components of samples, the same techniques and procedures can be applied to the analysis of other sample types and for examination of other sample components, such as nucleic acids, phospholipids, small organic molecules, etc. and their interactions.

Samples obtained from subjects include mixtures of proteins, including low molecular weight proteins, nucleic acids, phospholipids, small organic molecules, and other compounds that can be unique to a particular condition of interest, i.e., “biomarkers.”

As used herein, the term “protein” means any polymer comprising any of the 20 protein amino acids, regardless of its size. Although “protein” is often used in reference to relatively large polypeptides, and “peptide” is often used in reference to small polypeptides, usage of these terms in the art overlaps and varies. The term “protein” as used herein refers to peptides, polypeptides and proteins, unless otherwise noted. As used herein, the terms “protein”, “polypeptide,” and “peptide” are used interchangeably herein.

As used herein, the term “nucleic acid” refers to deoxyribonucleotides (DNA), ribonucleotides (RNA), including messenger RNA (mRNA) and microRNA (miRNA), and polymers thereof in either single or double stranded form.

As used herein, the term “phospholipids” means any amphipathic compounds arranged in a way that the ‘head’ is hydrophilic and the lipophilic ‘tail’ is hydrophobic.

As used herein, the term “small organic molecule” means any carbon based molecule that is not a polymer. In some embodiments, protein components of a sample are of interest.

The presence of and the expression level of specific proteins in a mixture of proteins found in a sample can be referred to as the proteomic profile of the sample. The proteomic profile of a sample obtained from a subject having a condition differs from the proteomic profile of a normal subject, i.e., condition-free subject. As such, information about a subject of unknown status (having condition vs. normal/lacking condition) can be obtained by comparing a thermogram generated from a sample obtained from the subject to a thermogram generated from a sample associated with a known status.

In some embodiments, proteins, nucleic acids, phospholipids, small organic molecules, and/or other components of a sample unique to a particular condition of interest (biomarkers) are of interest. The presence of and the expression level of specific biomarkers in a sample can be referred to as the biomarker profile of the sample. The biomarker profile of a sample obtained from a subject having a condition differs from the biomarker profile of a normal subject, i.e., condition-free subject. As such, information about a subject of unknown status (having condition vs. normal/lacking condition) can be obtained by comparing a thermogram generated from a sample obtained from the subject to a thermogram generated from a sample associated with a known status.

Such thermograms have many advantages, for example: they are easily obtained on unlabeled, underivitized, unfractionated biological samples; they consume only modest amounts of sample; they are obtained relatively quickly; they are based on rigorous, fundamental physical properties of proteins within the sample; they are quantitative, and reflect the exact protein composition of the sample; the procedures for obtaining thermograms are amenable to automated, high-throughput screening; and they provide a new window for viewing components of a biological sample based on thermal stability rather than on molecular weight and charge as is the case for electrophoresis and mass spectrometry.

The methods of the presently-disclosed subject matter make use of signature thermograms and standard thermograms. As used herein, the term signature thermogram refers to a thermogram generated using a particular sample of interest, or fraction of a sample of interest. The sample of interest is often a sample obtained from a particular subject.

In some embodiments, a method is provided for diagnosing or monitoring a condition of interest in a subject. In such embodiments, the signature thermogram can be a thermogram generated using a sample, or fraction thereof, obtained from the subject being diagnosed or monitored. In some embodiments, a method is provided for assessing a treatment program for a subject. In such embodiments, the signature thermogram can be a thermogram generated using a sample, or fraction thereof, obtained from the subject being whose treatment program is being assessed.

In some embodiments, a method of identifying biomarkers useful for diagnosing a condition of interest in a subject is provided. In such embodiments, the signature thermogram can be a thermogram particularly associated with the condition of interest, e.g., a sample or fraction of a sample from a subject known to have the condition of interest. In some embodiments, a method of screening a composition for use in treating a condition of interest in a subject is provided. In such embodiments, the signature thermogram can be a thermogram generated using a sample or fraction of a smaple obtained from the subject receiving the composition. In some embodiments, a method of screening a composition for plasma-protein interactions is provided. In such embodiments, the signature thermogram can be a thermogram generated using a plasma sample that has been contacted with the composition of interest, or a fraction thereof.

In some embodiments, it can be desirable to obtain multiple signature thermograms. In such embodiments, the multiple signature thermograms are generated using samples of interest, or fractions thereof, that are related in a particular manner. In such embodiments, samples of interest can be collected from the same subject (i.e., samples related in that they are obtained from the same subject) at different time points during the course of the treatment program.

As used herein, the term standard thermogram refers to a thermogram that is used as a reference to which a signature thermogram can be compared. A standard thermogram can be generated using a standard or control sample. A standard thermogram can be an average of multiple thermograms generated using multiple standard samples. For example, twenty (or another number of) standard samples can be obtained and a thermogram can be generated from each sample. The twenty generated thermograms could then be averaged to generate a standard thermogram.

In some embodiments, it can be desirable to provide a negative standard thermogram and/or a positive standard thermogram to which a signature thermogram can be compared. A negative standard thermogram is generated using a negative standard sample, or a fraction thereof. For example, a negative standard thermogram can be generated using a sample known to be associated with an absence of a condition of interest, e.g., a sample obtained from a subject known not to have a condition of interest. A positive standard thermogram is generated using a positive standard sample. For example, a positive standard thermogram can be generated using a sample known to be associated with a presence of a condition of interest, e.g., a sample obtained from a subject known to have a condition of interest.

A standard thermogram can be generated at a time point before, at a time point concurrent with or close to, or at a time point after the generation of a signature thermogram to which it will be compared. In some embodiments, it can be desirable to have a standard thermogram prepared to compare with various future-generated signature thermograms. In some embodiments, it can be desirable to provide a kit including one or more standard thermograms and instructions for generating signature thermograms for comparing with the one or more standard thermograms.

As noted herein, a thermogram can be obtained for a sample, or a fraction thereof. As used herein, the term “fraction” is used to describe a portion of a sample of interest, which is obtained by fractionating the sample. A sample can be divided into different portions, or fractions, using a variety of methods, i.e., fractionation methods, that will be known to those of ordinary skill in the art. For example, gel filtration can be used to fractionate a sample. Gel filtration chromatography separates protein mixtures on the basis of size and shape. Since plasma proteins, for example, vary greatly in the molecular weights and hydrodynamic shapes, gel filtration chromatography can fractionate a plasma sample into distinct component. Differential scanning calorimetry (DSC) of fractions emerging from a gel filtration column will reveal the thermograms of the protein components of those fractions.

As will be recognized by those skilled in the art, gel filtration is but one method by which a sample of interest can be fractionated. Additional fractionation methods include, but are not limited to the following: gel electrophoresis, chromatography, separation columns, immunoaffinity, centrifugation, mass spectroscopy, bioinformatic fractionation, or combinations thereof. Depending on the desired results, gel filtration chromatography, liquid chromatography (LC), LC-mass spectroscopy (LC-MS), affinity chromatography, and/or high pressure liquid chromatography (HPLC) can be desirable fractionation techniques. Other techniques that can bee desirable include, but are not limited to mass spectroscopy (MS) techniques, such as MS conducted using high-resolution LC-MS/MS, surface-enhanced laser desorption/ionization-time-of-flight (SELDI-TOF) MS, or matrix-assisted laser desorption/ionization-time-of-flight (MALDI-TOF) MS.

It is understood that fractionation can refer to separation by size, mass, shape, charge, thermal stability, or other physical quality of the sample components. The particular methods or techniques chosen will depend on the particular fluid to be fractionated. Suitable methods will be apparent to those skilled in the art.

By fractionating a sample and observing the thermograms of each fraction, it is possible to identify the component(s) in a sample whose thermogram(s) is (are) altered, e.g., a signature thermogram altered for a sample from a subjects having a condition of interest, as compared to standard thermogram. Since these components may be different because of substances bound thereto, the combination of fractionation and obtaining thermograms for each fraction provides a means of pinpointing the sample components to which potential biomarkers are bound.

When a sample is fractionated, and thermograms are obtained for multiple fractions of the sample, the thermograms can be described as a “series” of thermograms.

When a test sample is fractionated, and a control sample is similarly fractionated for purposes of comparison, certain fractions of the test sample and certain fractions of the control sample can be identified as “siblings.” For example, as will be understood by those skilled in the art, if a control sample and a test sample are fractionated by gel filtration chromatography, and fractions emerge from the gel filtration column at a fixed flow rate (e.g., 0.05 to 0.2 mL/min) and fractions of a fixed volume (e.g., 50-200 μL) are collected, then the first fraction of the control sample and the first fraction of the test sample can be said to be siblings, the second fraction of the control sample and the second fraction of the test sample can be said to be siblings, etc. As used herein, a standard thermogram that is a “sibling” of a signature thermogram is generated from a fraction of a control sample that is a “sibling” of a fraction of a test sample.

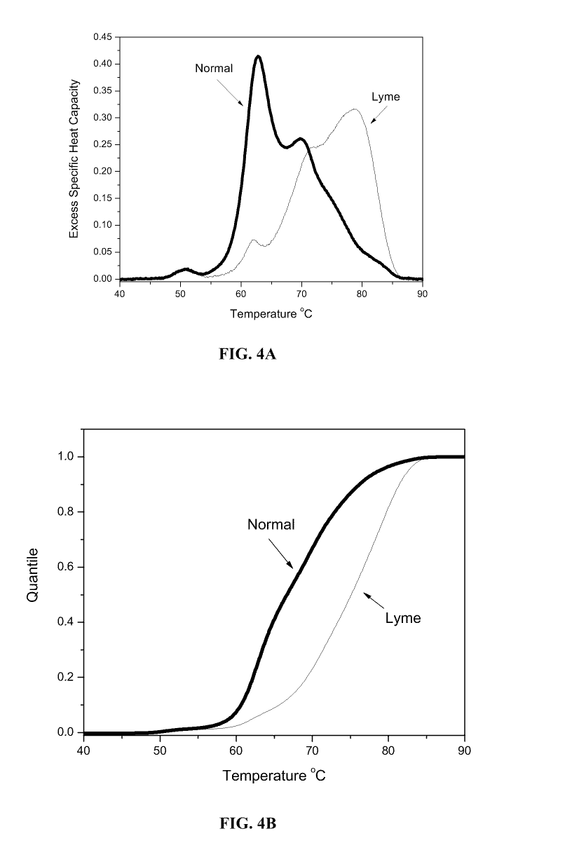

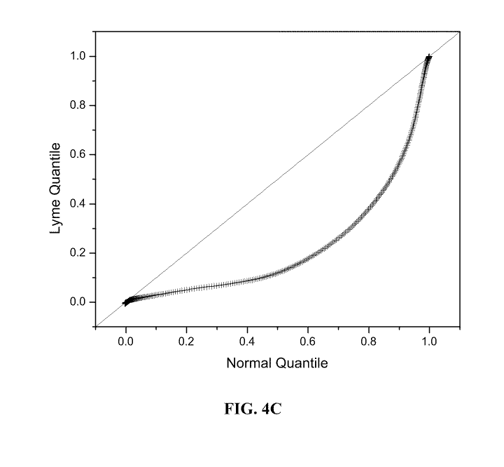

When comparing thermograms in accordance with methods of the presently-disclosed subject matter, they can be good simulations of one another or poor simulations of one another. When comparing thermograms, when a first thermogram is not a good simulation of a second thermogram, then it is a poor simulation of the second thermogram. A first thermogram is a good simulation of a second thermogram when it has substantial similarity to the second thermogram. In some embodiments, it is evident whether a first thermogram has substantial similarity to the second thermogram by inspection of the thermograms superimposed on one another, e.g., a signature thermogram superimposed on graphs of the standard(s). For example, FIG. 4A depicts a first normal thermogram (e.g., negative standard thermogram) and a second Lyme disease thermogram (e.g., signature thermogram) superimposed on one another. Upon inspection of the thermograms of FIG. 4A, it is evident that the first thermogram does not have substantial similarity to the second thermogram, i.e., poor simulation.

One of ordinary skill in the art can use his or her knowledge to make appropriate determinations of whether a substantial similarity can be found in particular situations. In some embodiments, substantial similarity can be found when each of the peaks of the first thermogram occur at about the same temperatures as each of the peaks of the second thermogram. In some embodiments, substantial similarity can be found when the peaks of the first thermogram occur at temperatures within one standard deviation of the peaks of the second thermogram. In some embodiments, substantial similarity can be found when the peaks of the first thermogram occur at temperatures within two standard deviations of the peaks of the second thermogram. In some embodiments, substantial similarity can be found when each of the peaks of the signature thermogram yield about the same heat capacity as the peaks of the standard thermogram. In some embodiments, substantial similarity can be found when the heat capacity of the peaks of the signature thermogram is within one standard deviation of the heat capacity of the peaks of the standard thermogram. In some embodiments, substantial similarity can be found when the heat capacity of the peaks of the signature thermogram is within two standard deviation of the heat capacity of the peaks of the standard thermogram.

In some embodiments, substantial similarity can be determined by application of published statistical procedures, for example, quantile-quantile plots (Lodder and Hieftje (1988)) can be used and/or a two-way Kolmogorov-Smirnov test can be used (Young (1977)). Briefly, for these tests, the thermogram must be converted to a quantile distribution. FIG. 4B depicts the quantile plots of the thermograms of FIG. 4A. Thermograms are converted to quantile distributions by the following steps: (1) the thermograms are baseline corrected and normalized thermograms are numerically integrated; (2) the integrated thermogram is normalized to 1.0; and (3) the resultant quantile plot thus consists of paired data points with temperature on the x-axis and normalized quantile values on the y-axis. To compare two thermograms, they must share common x values.

To construct a quantile-quantile plot, the quantile values derived from one thermogram is plotted against the quantile values derived from a second thermogram. FIG. 4C depicts a quantile-quantile plot generated using the quantile values of FIG. 4B, i.e., quantile for the first normal thermogram against the quantile for the second Lyme disease thermogram. If the two original thermograms are identical the paired data points will lie on a perfect straight line with a 45 degree angle from the origin. If the two original thermograms are not identical, points will deviate from the 45 degree straight line. As shown in FIG. 4C, the points for the Lyme disease quantile unambiguously and unacceptably deviate from the 45-degree straight line, indicating a poor simulation. In some embodiments, a first thermogram can be determined to be substantially similar to the second thermogram when the paired data points of the quantile-quantile plot lie on the 45 degree straight line, or have an acceptable deviation from the 45 degree straight line.

The same quantile values used to construct the quantile-quantile plot can be used to conduct a two-way Kolmogorov-Smirnov test, as implemented in standard statistical software packages and as is available online on service web sites (See, e.g., http://www.physics.csbsju.edu/stats/KS-test.html). The Kolmogorov-Smirnov test is designed to test the null hypothesis that two quantile distributions are not statistically different. The test returns a P-value for the confidence level with which the null hypothesis can be rejected. In this regard, in some embodiments, if the null hypothesis that the two quantile distributions are not statistically different (are good simulations) is rejected, it can be determined that the first thermogram is not substantially similar to the second thermogram. In some embodiments, the P-value is less than or equal to 0.5, 0.2, 0.1, 0.05, 0.02, 0.01, 0.005, 0.002, or 0.001.

By way of an example, when using the quantile values of FIG. 4B, a Kolmogorov-Smirnov test yields the results that the maximum between the cumulative distributions, D, is: 0.2028 with a corresponding P-value of less than 0.001, indicating that the null hypothesis that there is no difference between these quantile distribution can be rejected at the 99.999% confidence level, i.e., poor simulation.

In some embodiments of the presently-disclosed subject matter, a method of identifying biomarkers useful for diagnosis of a condition of interest in a subject is provided. The method of the presently-disclosed subject matter, which includes fractionation of samples, and generation of thermograms therefrom, provides a unique process for identifying useful biomarkers. For example, analysis of a fractionated sample by the presently-disclosed method can provide information about the relative concentration of all major component proteins, and other components, present in the sample; determination of the presence of binding interactions with the major component proteins, and other components, of the sample; and information about binding component(s) and evaluation of their binding constant(s). The presently-disclosed methods of identifying biomarkers incorporates the unique perspective that biomarkers can be detected by their influence on the thermodynamic stability of proteins in plasma and other biological samples.

DSC thermograms, as generated in accordance with the presently-disclosed subject matter, are highly sensitive to binding interactions, which is a unique and attractive feature of the technology. When a ligand recognizes and binds to a site on a protein in a sample, it stabilizes that protein with respect to thermal or chemical denaturation. Relative to the unligated protein, upon binding, the melting temperature is elevated or the concentration of denaturant required to unfold the protein is increased. Conversely, if a ligand were to selectively recognize some feature of the denatured protein, the melting temperature would be lowered. For DSC measurements of proteins, detailed and specific protocols for the analysis of ligand-induced shifts in denaturation thermograms have been published, and include closed form equations that may be applied to extract reliable binding constants for the ligand-protein interaction. The magnitude of melting temperature shifts for proteins in the presence of ligands can be dramatic (easily tens of degrees) and depends on the magnitude of the equilibrium binding constant and binding enthalpy.

The sensitivity of DSC thermogram technology to binding interactions is useful as both studies support the notion that, in the presence of a condition of interest, e.g., diseased states, low molecular weight proteins or circulating nucleic acids unique to the condition of interest (and therefore indicative of its presence), increase in concentration in biological samples, e.g., plasma, serum, CSF, etc. Such biomarkers form complexes with the more abundant proteins in the plasma (e.g., albumin and immunoglobulins) and can alter the denaturation thermogram profiles for the proteins which they bind. This binding produces characteristic changes in signature thermograms. Since the presently-disclosed subject matter is sensitive to such binding interactions in ways that current electrophoresis and mass spectrometry assay techniques are not, entirely new aspects of the plasma proteome can be divulged. Changes in signature thermograms resulting from binding of small proteins to a larger receptor are far more dramatic than changes in either mass or charge. Consequences of significant interactions with the major proteins are observed by alteration of the melting curve of proteins to which the biomarkers bind. A number of more standard biochemical characterization techniques can then be employed to identify and characterize biomarkers discovered in accordance with the presently-disclosed subject matter. For example, potential biomarkers in a sample can be isolated by separation techniques known to those of ordinary skill in the art, and further characterized by mass spectrometry.

In some embodiment, the method of identifying useful biomarkers includes: providing a test sample associated with the condition of interest; fractionating the test sample to obtain fractions of the test sample; generating a signature thermogram for each fraction of the test sample; comparing a signature thermogram to a sibling standard thermogram; and identifying as biomarkers any components of the sample that result in the signature thermogram being a poor simulation of the sibling standard thermogram.

In some embodiment of the presently-disclosed subject matter a system is provided for identifying useful biomarkers, which includes includes: means for fractionating a test sample to obtain fractions of the test sample; means for generating a signature thermogram for each fraction of the test sample; and means for comparing a signature thermogram to a sibling standard thermogram such that biomarkers can be identified.

In some embodiments, the standard thermogram is generated using a sample including a candidate biomarker. For example, control sample can be obtained and contacted with a candidate biomarker, such as a known protein, nucleic acid, phospholipid, small organic molecule, or another compound that can be unique to a particular condition of interest. In this regard, a candidate biomarker can be identified as an actual biomarker when the signature thermogram of a fraction of the test sample is a good simulation of the standard thermogram of the candidate biomarker.

In some embodiments, the method of identifying useful biomarkers further includes providing a control sample associated with an absence of the condition of interest; fractionating the control sample to obtain sibling fractions of the control sample; generating a sibling standard thermogram for each sibling fraction of the control sample; comparing a signature thermogram to a sibling standard thermogram for a sibling fraction of the control sample.

As will be understood by those skilled in the art, the biological sample provided for use in accordance with the presently-disclosed subject matter can be any appropriate biological sample that is suspected of containing a biomarker, such as a body fluid. Appropriate body fluids include, but are not limited to ascites fluid, blood, cerebral spinal fluid, serum, peritoneal fluid, plasma, saliva, senovial fluid ocular fluid, and urine. As will be understood by those skilled in the art, in some cases it can be desirable to select the type of sample being collected based on the selected condition of interest. For example, in some embodiments when the condition of interest is ALS, it can be desirable to obtain a cerebral spinal fluid sample. As is well understood by those skilled in the art, a control sample should be selected such that variables are limited. In this regard, for example, if the test sample is a plasma sample, it is preferred that the control sample is also a plasma sample. Similarly, if the test sample is a CSF sample, it is preferred that the control sample is also a CSF sample, etc.

In some embodiments, an obtained sample can be prepared in the following manner. A blood sample is drawn from the subject and plasma or serum is isolated from the blood using known methods. A small volume of about 100 μL, of plasma or serum is dialyzed at about 4° C. against a standard buffer (e.g., 10 mM potassium phosphate, 150 mM NaCl, 0.38% (w/v) sodium citrate, pH 7.5 for plasma; 10 mM potassium phosphate, 150 mM NaCl, pH 7.5 for serum). Dialyzed plasma or serum is filtered to remove particulates and then diluted about 25-fold into the standard buffer.

All samples can be prepared by essentially the same techniques, although buffer components and concentrations can vary with particular sample types. In any case, samples are equilibrated with a suitable buffer in order to compare with the buffer baseline of the reference. This can be achieved by dialysis or other suitable buffer exchange methods.”

The prepared sample is then fractionated using a desired technique. For example, in some embodiments, a fractionation technique is used that results in fractions including different size classes of proteins. In some embodiments, fractionating is conducted using gel filtration, gel electrophoresis, chromatographic fractionation, separation columns, immunoaffinity, centrifugation, mass spectroscopy, bioinformatic fractionation, or combinations thereof. In some embodiments, the fractionating is conduced using a chromatographic fractionation technique selected from: gel filtration chromatography, liquid chromatography (LC), LC-mass spectroscopy (LC-MS), affinity chromatography, and high pressure liquid chromatography (HPLC). In some embodiments, the fractionating is conduced using a mass spectroscopy technique selected from: high-resolution LC-MS/MS, surface-enhanced laser desorption/ionization-time-of-flight (SELDI-TOF) MS, and matrix-assisted laser desorption/ionization-time-of-flight (MALDI-TOF) MS.

The test sample and/or control sample fractions can then be used to generate one or more signature thermograms containing a protein composition pattern, or other component/biomarker composition pattern.

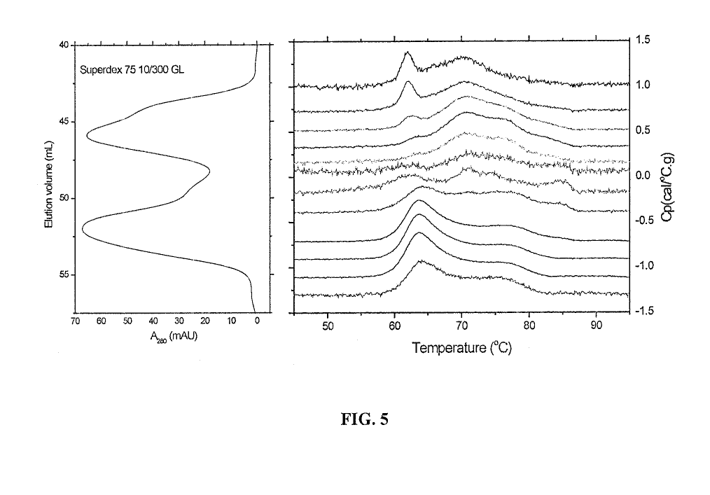

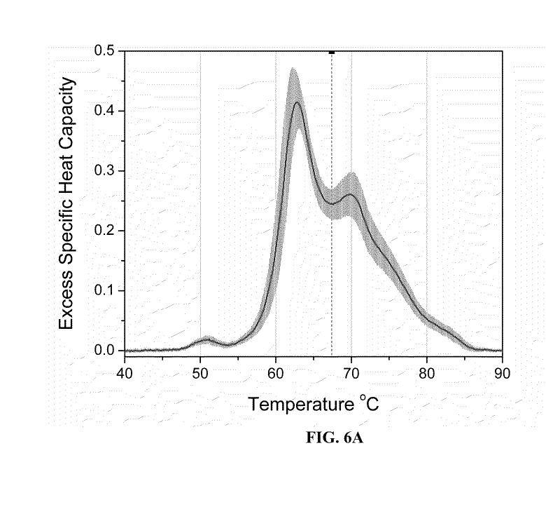

With reference to FIG. 5, a sample can be fractionated, as illustrated by the elution profile in the left panel, and then each fraction can be used to generate a signature thermogram, as illustrated by the series of signature thermograms in the right panel.

In some embodiments, a particular fraction will be of interest, and only one signature thermogram is generated. In some embodiments when a particular test fraction is of interest, and only one sibling standard thermogram is generated for purposes of comparing with the signature thermogram of interest. In some embodiments, a subset of fractions are of interest, and multiple signature thermograms are generated. In some embodiments when a particular subset of test fractions are of interest, and a subset of sibling control fractions are used to generate sibling standard thermograms for purposes of comparing with the signature thermograms of interest. In some embodiments, all of the fractions are of interest, and a complete series of signature thermograms are generated. In some embodiments when all of the test fractions are of interest, a complete series of sibling standard thermograms are generated for purposes of comparing with the series of signature thermograms.

Each fraction of interest is run on a differential scanning calorimeter (DSC) to obtain a thermogram for the sample. A differential scanning calorimeter (DSC) can be obtained from MicroCal, LLC (Northampton, Mass.), for example, the MicroCal, LLC VP Capillary Differential Scanning Calorimeter can be used. Any differential scanning calorimeter (DSC) with the requisite sensitivity, temperature range, scanning rate, and baseline stability could be used in accordance with the methods of the presently-disclosed subject matter. Examples of other instruments that would be suitable include: Calorimetric Sciences Corporation's N-DSCII Differential Scanning Calorimeter, TA Instruments Incorporated's Q2000 Differential Scanning Calorimeter, and Perkin Elmer Corporation's Diamond DSC Differential Scanning Calorimeter. Newly designed instruments that might become available with the requisite sensitivity, temperature range, scanning rate, and baseline stability could also be used to practice the methods of the presently-disclosed subject matter. For example, Energetic Genomics Corporation's 96-well differential scanning calorimeter that is under development, and for which a prototype instrument is available to the inventors, could be used to practice the methods of the presently-disclosed subject matter. Standard software and protocols can be used to obtain a thermogram for the sample with the selected DSC.

Using the MicroCal, LLC DSC as an example, a sample volume of approximately 0.4 mL is needed for liquid-handling for proper filling of the sample cell, although the effective cell volume is only approximately 0.133 mL. Each DSC run takes about 1-2 hours to complete. Total protein concentrations of the diluted sample can be determined by standard colorimetric, spectrophotometric, or refractometric methods. These concentrations can be used to normalize experimental thermograms to a g/L protein concentration scale. This normalized thermogram shows the “Excess Specific Heat Capacity” as a function of temperature for a plasma/serum sample (See, e.g., the dashed line thermogram of FIG. 3). Such a thermogram provides a specific signature for a particular sample that provides a snapshot of the protein composition of the sample.

When the desired signature and/or standard thermograms are obtained, they can be compared and assessments about the samples can be made. For example, if a signature thermogram of a first fraction of a test sample associated with a condition of interest is a poor simulation of sibling negative standard thermogram of a first fraction of a control sample, it is indicative of a unique component associated with the condition of interest being present in the first fraction of the test sample. This unique component, for example, could be interacting with a particular plasma protein found in that fraction, e.g., an albumin fraction. The fraction identified as containing the unique component can be further examined to identify the unique component, which can be useful as a biomarker for the condition of interest. For example, in some embodiments the fraction can be analyzed using mass spectrometry to identify the unique component of the fraction.

In some embodiments, test samples and negative control samples are fractionated, as described herein, and thermograms are generated for the various resulting fractions. The surface defined by the two dimensions of elution volume and temperature reveals the unique components of the test samples, and fractions thereof, relative to the negative control samples, and sibling fractions thereof.

In some embodiments, it can be useful samples and/or fractions thereof with a denaturant (e.g., urea, guanidine HCL) and to obtain thermograms as a function of added denaturant concentration. Such a method can uncover additional distinctions between components of samples, and fractions thereof, associated with a condition of interest, and negative control samples, and fractions thereof, associated with a lack of a condition of interest. In some embodiments, unfractionated plasma test samples and negative control samples are subjected to various concentrations of denaturant, and thermograms are generated. The surface defined by two dimensions of denaturant concentration and termperture reveals the unique components of the test samples relative to the negative control samples. Binding interactions stabilize native conformations and can be elevated at the concentration of denaturant required for unfolding.

In some embodiments of the presently-disclosed subject matter, a method of diagnosing or monitoring a condition of interest in a subject is provided. In some embodiments, a method of diagnosing or monitoring a condition of interest in a subject includes, providing a test sample obtained from the subject, fractionating the test sample to obtain fractions of the test sample; generating a signature thermogram for at least one fraction of the test sample; comparing at least one signature thermogram with a standard thermogram; and identifying the subject as having the condition of interest or lacking the condition of interest. In some embodiments, the method further includes providing a control sample; fractionating the control sample to obtain sibling fractions of the control sample; generating a sibling standard thermogram for at least one sibling fraction of the control sample; comparing the signature thermogram with the at least one sibling standard thermogram; and identifying the subject as having the condition of interest or lacking the condition of interest.

In some embodiments of the presently-disclosed subject matter, a system for diagnosing or monitoring a condition of interest in a subject is provided, which includes means for fractionating a test sample to obtain fractions of the test sample; means for generating a signature thermogram for at least one fraction of the test sample; and means for comparing at least one signature thermogram with a standard thermogram, such that it can be determined whether the signature thermogram is a good simulation or a poor simulation of the standard thermogram.

As will be understood by those skilled in the art, the sample obtained from the subject can be any appropriate biological sample, such as a body fluid. Appropriate body fluids include, but are not limited to ascites fluid, blood, cerebral spinal fluid, serum, peritoneal fluid, plasma, saliva, senovial fluid, ocular fluid, and urine. As will be understood by those skilled in the art, in some cases it can be desirable to select the type of sample being collected based on the selected condition of interest. For example, in some embodiments when the condition of interest is ALS, it can be desirable to obtain a cerebral spinal fluid sample.

As will be understood by those skilled in the art, and as noted hereinabove, when a control sample is provided, it can be a positive control sample or a negative control sample. For example, a positive control sample can be a biological sample that is obtained from a subject known to have the condition of interest, while a negative control sample can be a biological sample that is obtained form a subject known to be normal or free of the condition of interest. As is well-understood by those skilled in the art, a control sample should be selected such that variables are limited. In this regard, for example, if the test sample is a plasma sample, it is preferred that the control sample is also a plasma sample. Similarly, if the test sample is a CSF sample, it is preferred that the control sample is also a CSF sample, etc.

The obtained sample can be prepared as described herein, and also fractionated as described herein.

The test sample and/or control sample fractions can then be used to generate one or more signature thermograms containing a protein composition pattern, or other component/biomarker composition pattern. In some embodiments, a particular fraction will be of interest, and only one signature thermogram is generated. In some embodiments when a particular test fraction is of interest, and only one sibling standard thermogram is generated for purposes of comparing with the signature thermogram of interest. In some embodiments, a subset of fractions are of interest, and multiple signature thermograms are generated. In some embodiments when a particular subset of test fractions are of interest, and a subset of sibling control fractions are used to generate sibling standard thermograms for purposes of comparing with the signature thermograms of interest. In some embodiments, all of the fractions are of interest, and a complete series of signature thermograms are generated. In some embodiments when all of the test fractions are of interest, a complete series of sibling standard thermograms are generated for purposes of comparing with the series of signature thermograms.

Each fraction of interest is run on a differential scanning calorimeter (DSC) to obtain a thermogram for the sample, as described herein.

Once at least one signature thermogram is generated, it can be compared to a standard thermogram or another signature thermogram. To minimize uncontrolled variables, the test sample used to generate the signature thermogram should be prepared in the same manner as the control sample used to generate the standard thermogram, or the sample used to generate another signature thermogram. Similarly, the calorimeter, software, and protocols used to generate the thermograms that are to be compared should be substantially the same.

The standard thermogram can be a negative standard thermogram, in that it is associated with an absence of the condition of interest. The negative standard thermogram can be generated using a sample obtained from a subject who is “normal,” i.e., condition-free. In some cases the sample can have been obtained from the subject being diagnosed or monitored at a time when that subject was known to be condition-free. The standard thermogram can also be a positive standard thermogram, in that it is associated with a presence of the condition of interest. The positive standard thermogram can be generated using a sample obtained from a subject who has the condition of interest. In some cases the sample can have been obtained from the subject being diagnosed or monitored at a time when that subject was known have the condition. In some embodiments, the signature thermogram can be compared to both a negative standard thermogram and a positive standard thermogram.

In some embodiments, the subject can be identified as having the condition of interest when the signature thermogram of a fraction of the test sample is compared to a negative standard thermogram, and is found to be a poor simulation of the negative standard thermogram.

In some embodiments, the subject can be identified as having the condition of interest when the signature thermogram of a fraction of the test sample is compared to a positive standard thermogram, and is found to be a good simulation of the positive standard thermogram.

In some embodiments, the subject can be identified as having the condition of interest when the signature thermogram is compared to a positive standard thermogram and a negative standard thermogram, and is found to be a good simulation of the positive standard thermogram and a poor simulation of the negative standard thermogram

In some embodiments, in which multiple signature thermograms and standard thermorgrams are generated, the subject can be identified as having the condition of interest when at least one of the signature thermograms of a fraction of the test sample is compared to a negative standard thermogram, and is found to be a poor simulation of the negative standard thermogram.

In some embodiments, in which multiple signature thermograms and standard thermorgrams are generated, the subject can be identified as having the condition of interest when at least one of the signature thermograms of a fraction of the test sample is compared to a positive standard thermogram, and is found to be a good simulation of the positive standard thermogram.

In some embodiments, in which multiple signature thermograms and standard thermorgrams are generated, the subject can be identified as having the condition of interest when at least one of the signature thermograms is compared to a positive standard thermogram and a negative standard thermogram, and is found to be a good simulation of the positive standard thermogram and a poor simulation of the negative standard thermogram.

In some embodiments, the subject can be identified as lacking the condition of interest when the signature thermogram of a fraction of the test sample is compared to a negative standard thermogram and is found to be a good simulation of the negative standard thermogram.

In some embodiments, the subject can also be identified as lacking the condition of interest when the signature thermogram of a fraction of the test sample is compared to a positive standard thermogram and is found to be a poor simulation of the positive standard thermogram.

In some embodiments, the subject can also be identified as lacking the condition of interest when the signature thermogram of a fraction of the test sample is compared to a negative standard thermogram and a positive standard thermogram, and is found to be a good simulation of the negative standard thermogram and a poor simulation of the positive standard thermogram.

In some embodiments, in which multiple signature thermograms and standard thermorgrams are generated, the subject can be identified as lacking the condition of interest when all of the signature thermograms of the fraction of the test sample are compared to sibling negative standard thermograms, and are found to be good simulations of the negative standard thermograms.

In some embodiments, in which multiple signature thermograms and standard thermorgrams are generated, the subject can be identified as having the condition of interest when all of the signature thermograms of the fractions of the test sample are compared to sibling positive standard thermograms, and is found to be a poor simulation of the positive standard thermograms.

In some embodiments, in which multiple signature thermograms and standard thermorgrams are generated, the subject can be identified as having the condition of interest when all of the signature thermograms are compared to sibling positive standard thermograms and sibling negative standard thermogram, and are found to be poor simulations of the positive standard thermograms and a good simulations of the negative standard thermograms.

In some embodiments, the subject can be identified as having a condition, albeit unidentified for the time being, when the signature thermogram is found to be a poor simulation of the negative standard thermogram. Upon such a finding, the signature thermogram can then be compared to positive standard thermograms associated with conditions of interest in order to make a diagnosis.

In some embodiments, the signature thermogram can be compared to multiple positive standard thermograms, e.g., a database including multiple positive standard thermograms, each positive standard thermogram being associated with a particular condition of interest. As will be recognized by those of ordinary skill in the art, where standard thermograms are used that are previously generated, e.g., standard thermograms found in a database, care should be taken to minimize variables. As such, it is preferred that the standard thermogram that is selected for comparison be one that was generated using a fraction of a sample of the same type, fractionated by the same method, and being of a “sibling” fraction as the test fraction used to generate the signature thermogram. The positive standard thermogram obtained under the parameters that most resembles those under which the signature thermogram was obtained can be selected. The subject can be identified as having the condition associated with the positive standard thermogram that most resembles the signature thermogram. In some embodiments, the method can be useful to distinguish between two conditions having initial symptoms that are difficult to distinguish; for example, in some embodiments, the method can be used to distinguish multiple sclerosis and ALS in a subject.

As will be understood by those of ordinary skill in the art, it can sometimes be desirable to obtain multiple samples from the subject at various time points, in order to monitor the condition of interest. For example, in some embodiments, a second sample can be obtained from the subject at a time point after the first sample is obtained. The second sample can be fractionated and a signature thermogram can be generated from one or more fractions of the second sample. The first signature thermogram, or series of first signature thermograms, can be compared to the second signature thermogram, or series of second signature thermograms. The condition of interest can be identified as changed when the second sibling signature thermogram is a poor simulation of the first signature thermogram. The condition of interest can be identified as unchanged when the second sibling signature thermogram is a good simulation of the first signature thermogram.

As such, in some embodiments, the method of diagnosing or monitoring a condition of interest further includes providing a second test sample obtained from the subject at a time point after the test sample is obtained; fractionating the second test sample to obtain fractions of the second test sample; generating a signature thermogram for each fraction of the second test sample; comparing a signature thermogram of the second test sample with a standard thermogram, and/or a signature thermogram of the first test sample; and identifying the subject as having the condition of interest or lacking the condition of interest, and/or having a change or having no change in status associated with the condition of interest.

In some embodiments, the method of diagnosing or monitoring a condition of interest further includes providing a second test sample obtained from the subject at a time point after the test sample is obtained; fractionating the second test sample to obtain fractions of the second test sample; generating signature thermograms for each fraction of the second test sample; comparing a signature thermogram of the second test sample with a sibling standard thermogram of the control sample, and/or a sibling signature thermogram of the first test sample; and identifying the subject as having the condition of interest or lacking the condition of interest; or having a change or having no change in status associated with the condition of interest.

In some embodiments, the second signature thermogram (including second sibling signature thermogram, or second series of signature thermograms) can also be compared to a negative standard thermogram (including sibling negative standard thermogram, or a series of negative standard thermograms). If the second signature thermogram is a good simulation of the negative standard thermogram, for a subject that had previously been identified as having a particular condition, the subject can be identified as having improved to the point of lacking the condition. In some embodiments, the second signature thermogram can also be compared to various positive standard thermograms (including sibling positive standard thermograms, or a series of positive standard thermograms) associated with different stages of a particular condition. In this regard, it can be determined whether the condition is progressing, i.e., becoming more severe, or regressing, i.e., improving.

The presently-disclosed subject matter also includes a method of assessing efficacy of a treatment program for a subject having a condition of interest, or being at risk for developing the condition of interest. As used herein, a treatment program includes a plan for treating a subject or providing treatment to a subject. As used herein, the terms treatment or treating relate to any treatment of a condition of interest, including but not limited to prophylactic treatment and therapeutic treatment. As such, the terms treatment or treating include, but are not limited to: preventing the development of a condition of interest; inhibiting the progression of a condition of interest; arresting or preventing the development of a condition of interest; reducing the severity of a condition of interest; ameliorating or relieving symptoms associated with a condition of interest; and causing a regression of the condition of interest or one or more of the symptoms associated with the condition of interest. As will be understood by those of ordinary skill in the art, a treatment program can differ depending on the condition of interest and the subject being treated. A treating physician can select a particular treatment program based on the condition of interest, and the particular subject being treated. Depending on the situation, a treatment program could include, for example, administering a treatment composition or a series of treatment compositions, administering a radiation treatment, prescribing an altered diet, prescribing a particular exercise regimen, prescribing low activity or rest, a combination thereof, etc.

In some embodiments, a method of assessing a treatment program for a subject includes the following: providing a first sample obtained from the subject at a first time point of interest, e.g., prior to the initiation of the treatment program; fractionating the first test sample to obtain a first series of fractions; generating a first series of signature thermograms for the first series of fractions of the first test sample; providing a second test sample obtained from the subject at a second time point of interest, e.g., after the initiation of the treatment program; fractionating the second test sample to obtain a second series of fractions; generating a second series of signature thermograms for the second series of fractions of the second test sample; comparing the first series of signature thermograms to the second series of signature thermograms; and identifying the presence or absence of a change in the condition of interest.

The first sample can be obtained from the subject before initiation of the treatment program, or at another time point of interest that will service as a base-line by which the treatment program will be assessed. The first sample is fractionated, as described herein, and the fractions are used to generate a first series of signature thermograms. It is contemplated that in some embodiments, a particular fraction or subset of fractions will be of interest, and it is possible that only the particular fraction or subset of fractions will be used to generate signature thermograms. In other embodiments, it will be desirable to obtain a complete series of first signature thermograms.

In some embodiments, the subject has a condition of interest when the first sample is collected. In some embodiments, the subject does not have a condition of interest, but there is otherwise a reason for receiving a treatment program, as will be understood by those of ordinary skill in the art. For example, a subject lacking a condition of interest, but having a risk for obtaining the condition of interest could receive a treatment program, the efficacy of which can be assessed using the method of the presently-disclosed subject matter.

The second sample is obtained from the subject at a second time point of interest, e.g., following the initiation of the treatment program. The treatment program can include, for example, administration of a treatment composition and the second sample can be obtained after the subject has been receiving the treatment composition for a day, week, month, or other time period of interest. For another example, the treatment program can include providing radiation treatment and the second sample can be obtained after the subject has been receiving the radiation treatment for a specific period of time. In any event, the second sample is generally obtained at a time point of interest after the treatment program has been initiated. Additional samples can be obtained at different time points of interest to generate a time course describing the effect of the treatment program on the subject.

The second sample is fractionated and used to generate a sibling series of second signature thermograms associated with the treatment program of the subject. It is contemplated that in some embodiments, a particular fraction or subset of fractions will be of interest, and it is possible that only the particular fraction or subset of fractions will be used to generate signature thermograms. In other embodiments, it will be desirable to obtain a complete series of second signature thermograms. Generally, it will be desirable to second signature thermograms that are siblings of each generated first signature thermogram, such that the sibling second signature thermograms can be compared to each of the first signature thermograms.

The signature thermograms are generated by running the samples on a differential scanning calorimeter (DSC), as described herein. Once the signature thermograms are generated, they can be compared to one another. To minimize uncontrolled variables, the sample used to generate the first signature thermogram (or series of first signature thermograms) should be prepared in the same manner and be of the same type as the sample used to generate the second signature thermogram (or series of second signature thermograms). Similarly, the calorimeter, software, and protocols used to generate each of the thermogram should be substantially the same.

When the signature thermograms are compared, the treatment program can be identified as having not changed the condition of the subject (i.e., indicating absence of a change in status, or maintaining the status of the subject) when each second signature thermgram of the fractions of the second sample is a good simulation of the sibling first signature thermograms of the sibling fractions of the first sample.

When the signature thermograms are compared, the treatment program can be identified as affecting a change in the condition of the subject (i.e., indicating a change in status of the subject) when at least one second signature thermogram of the fractions of the second sample is a poor simulation of the sibling first signature thermogram of the sibling fraction of the first sample.

As will be understood by those of ordinary skill in the art, depending on the goal of the treatment program, an absence or a presence of a change can be indicative of an effective or an ineffective treatment program. As such, the determination of whether the presence or absence of a change is indicative of an effective treatment program will differ depending on the goal of the treatment program.

In some embodiments, when there is an absence of a change, the treatment program can be identified as an effective treatment program. In some embodiments, when there is an absence of a change, the treatment program can be identified as an ineffective treatment program. For example, if a prophylactic treatment program is administered to a subject lacking a condition of interest, with a goal of preventing an onset of the condition of interest, an absence of a change in the condition of the subject can be indicative of an effective (successful) treatment program. For another example, if a therapeutic treatment program is administered to a subject having a condition of interest, an absence of a change in the condition of the subject can be indicative of an effective treatment program if the goal is to prevent progression of the condition, or an ineffective treatment program if the goal is to cause a regression of the condition.

In some embodiments, when there is a presence of a change, the treatment program can be identified as an effective treatment program. In some embodiments, when there is a presence of a change, the treatment program can be identified as an ineffective treatment program. For example, in some embodiments, a prophylactic treatment program is administered to a subject who initially lacked a condition of interest; in such embodiments, a change in the condition can be indicative of an ineffective treatment program.

In some embodiments, it is apparent by inspecting the thermograms whether a change is indicative of an effective or an ineffective treatment program, e.g., change indicative of a regression of a condition, or a progression of a condition, as will be understood by those of ordinary skill in the art. In some embodiments, it can be desirable to additionally compare the signature thermogram to one or more standard thermograms. For example, in some embodiments a treatment program is administered to a subject who initially had a condition of interest; in such embodiments, a change in the condition can be indicative of either a regression or a progression of the condition. In such cases, as will be understood by those of ordinary skill in the art, it can be useful to additionally compare the second signature thermogram to one or more standard thermograms. For example, if the second signature thermogram is a good simulation of a negative standard thermogram, then the change can be indicative of a regression. In some embodiments, it can be useful to compare the second signature thermogram to a multiple positive standard thermograms, each associated with a particular stage of the condition of interest. Such comparisons can also provide information about whether a change in the condition is indicative of a progression or a regression of the condition.

The presently-disclosed subject matter also includes a method of screening for a composition useful for treating a condition of interest. In some embodiments, the method includes: interacting a sample associated with the condition of interest with a candidate composition; fractionating the sample to obtain a series of fractions; generating a series of signature thermograms for the series of fractions; comparing the series of signature thermograms to sibling standard thermograms; and determining the utility of the candidate composition.

With regard to the step of interacting the sample associated with the condition of interest with a candidate composition, in some embodiment, the candidate composition can be administered to an infected subject. The subject can be any appropriate test subject, for example, a mouse, a rat, a rabbit, or another appropriate test subject. In some embodiments, the candidate treatment composition can be administered to a subject that is a model for a condition of interest, e.g., mouse model for a particular condition. The candidate composition can be administered by any appropriate method, depending on the characteristics of the composition being screened. A sample, e.g., body fluid sample, can then be obtained from the test subject for use in generating the signature thermogram. In some embodiments, the step of interacting a sample associated with the condition of interest with a candidate treatment composition includes administering the candidate treatment composition to cells in culture, which cells have been infected with or are otherwise associated with the condition of interest. A sample can then be extracted from the cells for use in obtaining fractions and generating the series of signature thermograms. The fractionating can be conducting using techniques identified herein. The signature thermograms can be generated using a differential scanning calorimeter (DSC).

Once the signature thermogram (or series of signature thermorgrams) is generated, it can be compared to a sibling standard thermogram (or series of sibling standard thermograms). To minimize uncontrolled variables, the sample used to generate the signature thermogram should be prepared in the same manner and obtained from the same species as the sample used to generate the standard thermogram. Similarly, the calorimeter, software, and protocols used to generate the signature thermogram should be substantially the same as those used to generate the standard thermogram.

The standard thermogram can be a negative standard thermogram, in that it is associated with an absence of the condition of interest. The negative standard thermogram can be generated using a sample fraction(s) associated with an absence of the condition of interest, e.g., fractions of a sample obtained from a subject who is “normal,” or condition-free. In some embodiments, the negative standard sample can be obtained from a subject administered the candidate treatment composition, in which case it is obtained prior to the infection of the subject and prior to administration of the candidate treatment composition.

The standard thermogram can also be a positive standard thermogram, in that it is associated with a presence of the condition of interest. In some embodiments, the positive standard thermogram can be generated using a sample fraction(s) obtained from a subject who has the condition of interest. In some embodiments, the positive standard sample can be obtained from the subject administered the candidate treatment composition, in which case it is obtained after the subject is infected and prior to administration of the candidate treatment composition.

In some embodiments, the signature thermogram is a good simulation of the sibling negative standard thermogram associated with an absence of the condition of interest, and the candidate treatment composition can be identified as being useful.

In some embodiments, the signature thermogram is a good simulation of the sibling positive standard thermogram associated with a presence of the condition of interest. It can then be determined whether the candidate treatment composition is either useful for preventing a progression of the condition, or is ineffective if the goal is to cause a regression of the condition.

In some embodiments, the signature thermogram is a poor simulation of the sibling negative standard thermogram and/or a poor simulation of the sibling positive standard thermogram. It can then be determined whether the candidate treatment composition is either useful for causing a regression of the condition, useful for preventing a progression of the condition, or is ineffective, i.e., not treatment affected, or causes a progression of the condition.

In order to make the determination of whether the candidate treatment composition is useful for causing a regression of the condition, useful for preventing a progression of the condition, or is ineffective, it can be desirable to obtain multiple samples collected over time, for use in generating a multiple of signature thermograms (or multiple series of signature thermograms). The multiple of signature thermograms generated from samples collected over time and fractionated can be compared to identify any changes. In some embodiments, it is apparent by inspecting multiple sibling signature thermograms whether a change is indicative of an effective or an ineffective treatment program. For example, if the multiple sibling of signature thermograms of sibling fractions of samples collected over time display a trend towards a good simulation of the sibling negative standard thermogram, then it can be determined that the candidate treatment composition causes a regression of the condition. For another example, if the multiple sibling of signature thermograms of sibling fractions of samples collected over time display no change, then it can be determined that the candidate treatment composition prevents a progression of the condition. For another example, if the multiple sibling of signature thermograms of sibling fractions of samples collected over time display a trend towards a good simulation of the sibling positive standard thermogram, then it can be determined that the candidate treatment composition neither causes a regression of the condition nor prevents a progression of the condition, i.e., ineffective.

In some embodiments, it can be desirable to additionally compare the signature thermogram (or series of signature thermograms) to one or more sibling standard thermograms. In some embodiments, the signature thermogram can be compared to one or more sibling positive standard thermograms associated with different stages of a condition of interest. For example, if the condition of interest is cervical cancer, standard thermograms associated with moderate cervical dysplasia (CIN II), early stage cervical cancer, and stage IVB cervical cancer can be provided. The signature thermograms can be used to determine whether the candidate treatment composition affects a regression of the cervical cancer from stage IVB cervical cancer, to early stage cervical cancer, to moderate cervical dysplasia; a progression from moderate cervical dysplasia, to early stage cervical cancer, to stage IVB cervical cancer; or no change. In some embodiment where the condition of interest is a brain cancer, standard thermograms associated with grade 1 astrocytoma, grade 2 astrocytoma, grade 3 astrocytoma, and grade 4 astrocytoma (also referred to as glyoblastoma mutiforme) can be provided. The signature thermograms can be used to determine whether the candidate treatment composition affects a regression in the brain cancer, a progression in the brain cancer, or no change.

In some embodiments, the candidate treatment composition can be administered to a test subject before the test subject has been infected with the condition of interest. The subject can then be infected, samples obtained, samples fractionated, and thermograms generated. The thermograms can be compared to determine the ability of the candidate treatment composition to prevent or inhibit an onset or progression of a condition of interest.

The presently-disclosed subject matter further includes a method of screening a composition, e.g. candidate drug or treatment, for protein interactions, to identify and/or monitor the capacity of the composition to interact with protein. In some embodiments, the method includes: interacting the composition with a first plasma sample; fractionating the first plasma sample to obtain a first series of fractions; generating a first series of signature thermograms for the first series of fractions; comparing the first series of signature thermograms to sibling negative standard thermograms associated with an absence of plasma protein interactions; and/or a sibling second series of signature thermogram generated using a second series of fractions from a second plasma sample not interacted with the composition; and identifying the composition as lacking substantial plasma protein interactions when the first series of signature thermograms are good simulations of the sibling negative standard thermograms, and/or the sibling second series of signature thermograms.

In some embodiments, the thermogram containing a protein composition pattern associated with an absence of protein interactions can be a negative standard thermogram. In some embodiments, the thermogram containing a protein composition pattern associated with an absence of protein interactions can be a second signature thermogram generated using a second sample not interacted with the composition.

In some embodiments, the sample is a plasma sample or a serum sample. In such embodiments, the method can be used to identify and/or monitor capacity of composition, e.g., candidate drug, to bind serum albumin and/or other serum or plasma protein interactions. During drug development and efficacy studies, it can be desirable to identify and monitor interactions between a compound of interest (e.g., drug candidate) and components of plasma. For example, it will be appreciated by those of ordinary skill in the art that it can be desirable to identify and/or monitor a compound of interest for binding to serum albumin.

The presently-disclosed subject matter is further illustrated by the following specific but non-limiting examples. The following examples may include compilations of data that are representative of data gathered at various times during the course of development and experimentation related to the present invention.

### EXAMPLES

### Reproducible Thermogram for Normal Plasma

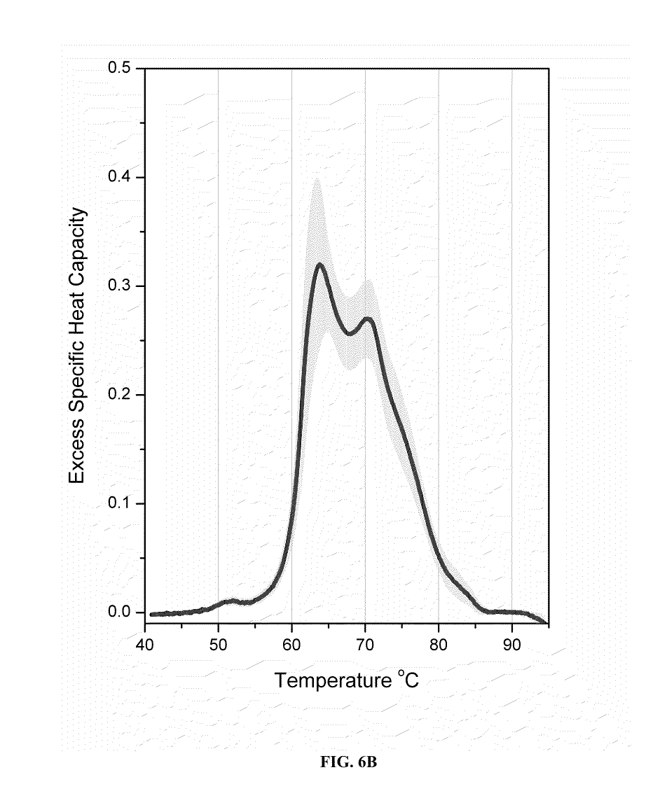

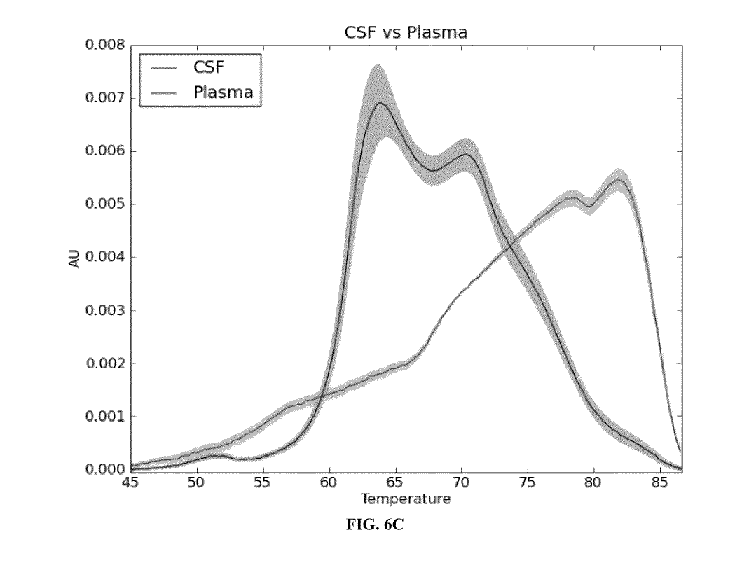

FIG. 6A shows an average thermogram obtained from plasma samples from 15 normal subjects. FIG. 6B shows an average thermogram obtained from plasma samples from 100 normal subjects. FIG. 6C shows an average thermogram obtained from plasma samples from normal subjects, and an average thermogram obtained from CSF samples from normal subjects. The thermograms displays multiple peaks and shoulders, yet are surprisingly simple, given the complexity of the plasma proteome. The average thermogram is shown as the black trace, and the standard deviation from the mean appears as the shaded regions of FIGS. 6A-6C. The standard deviation of the data is low, and is comparable to the range in values observed in normal subjects for the concentrations of individual plasma proteins (Craig (2004)). Human serum albumin, for example, has a normal reference range of approximately 35 to 55 g/L, dependent on age and gender (Craig (2004)). This analysis indicates that thermograms from normal subjects are highly reproducible. As noted herein, the thermograms for samples associated with various conditions of interest all deviate beyond the range of normal values of the thermogram of FIGS. 6A-6C, and their patterns must be considered to be significantly different from normal.

The average normal thermogram in FIG. 6A shows clear peaks at 50.8, 62.8 and 69.8° C. The area under the thermogram is 5.02±0.23 cal g⁻¹, and defines the specific enthalpy for the denaturation of normal plasma over the range 45-90° C. The first moment of the thermogram with respect to the temperature axis is 67.4±0.8° C. The sample size used in these studies is appropriate for exploratory preclinical studies, and, indeed, is on par with the numbers expected for a Phase I clinical trial (Motulsky (1995)).

Normal plasma thermogram is the weighted sum of the denaturation of individual plasma proteins. Applicants hypothesized that the thermograms seen in FIGS. 6A and 6B arises from the denaturation of the individual proteins within plasma, and represents the sum of individual protein denaturation reactions weighted according to their concentrations within plasma.

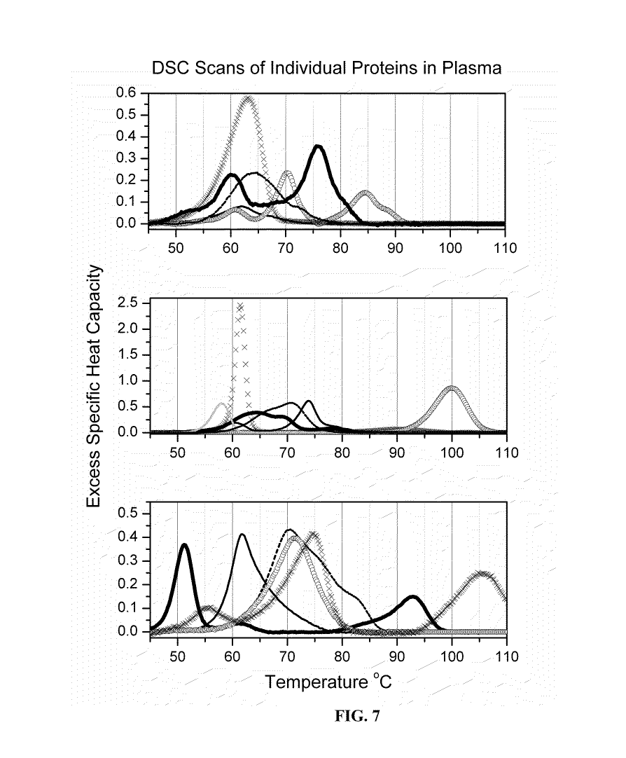

This hypothesis was tersted. With reference to FIG. 7, individual thermograms for the denaturation of the sixteen (16) most abundant plasma proteins were determined. FIG. 7 includes a series of thermograms of individual purified plasma proteins. The top panel shows superimposed thermograms for α₁-antitrypsin (black), transferrin (circles), α₁-acid glycoprotein (dashed), complement C3 (thick black), and c-reactive protein (crosses). The middle panel shows thermograms for haptoglobin (crosses), prealbumin (circles), α₂-macroglobulin (thick black), complement C4 (black), α₁-antichymotrypsin (gray), and IgM (dashed). The bottom panel shows thermograms for albumin (black), IgG (dashed), fibrinogen (thick black), IgA (circles), and ceruloplasmin (crosses). These thermograms display a range of denaturation temperatures, and differences in the complexities of their denaturation reactions. Many of these thermograms show multiple peaks, indicative of complex denaturation reactions, while other thermograms are consistent with simple two-state melting behavior.

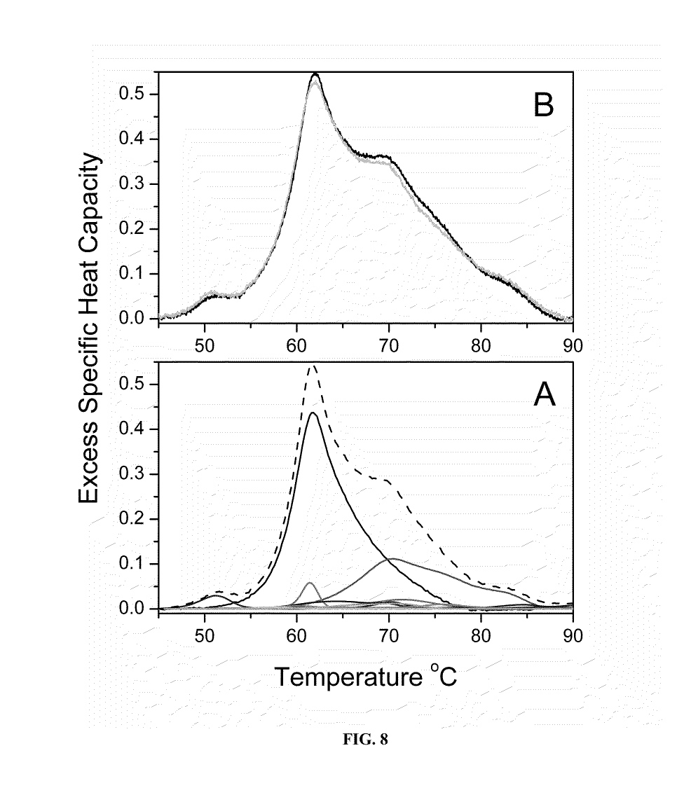

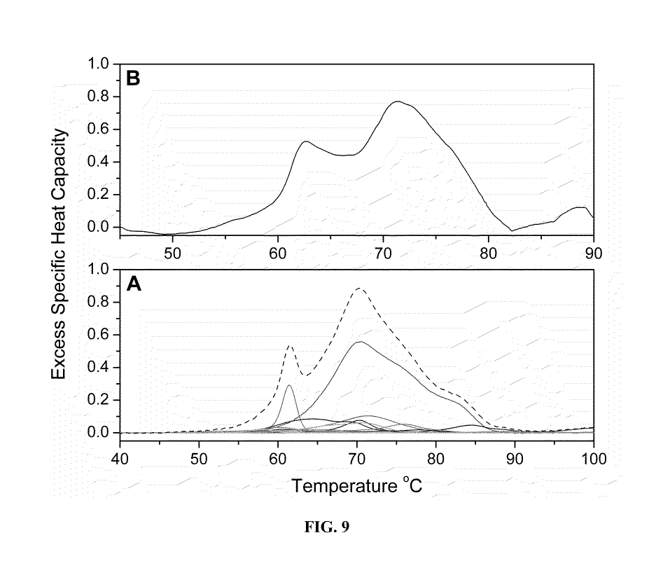

FIG. 8 (Panel A) shows the calculated plasma thermogram obtained by simple summation of the individual thermograms for the 16 most abundant plasma proteins after weighting their contribution according to their known average concentrations in normal plasma (Craig (2004)). Multicomponent analysis was used. A tacit assumption in this exercise is that there are no interactions among these proteins that might alter their thermal denaturation. The resultant shape of the calculated thermogram mimics that of the experimental one seen in FIG. 8, in support of the Applicants' hypothesis.

Referring now to FIG. 8 (Panel B), as a second test, mixtures of pure individual plasma proteins were prepared, and their thermograms determined by DSC. A mixture containing the 16 most abundant plasma proteins at their average concentrations found in normal plasma yields a thermogram whose shape mimics that of actual plasma (black curve of FIG. 8 (Panel B)). A mixture with only the four (4) major components (HSA, IgG, fibrinogen and transferrin) yields a thermogram that closely matches the observed normal, but which lacks subtle features (gray curve of FIG. 8 (Panel B)).

The data presented in FIG. 8 show that the normal thermogram is dominated by contributions from those four proteins. The small peak at 50.8° C. can be unambiguously assigned to a transition in fibrinogen. The major peak at 62.8° C. primarily reflects the denaturation of unligated HSA, with a contribution from haptoglobin. The peak a 69.8° C. and the shoulders at higher temperature arise primarily from IgG.

Thermograms of HSA-depleted serum. FIG. 9 shows the results from experiments in which albumin was removed from serum by affinity chromatography. (Serum differs from plasma primarily by the absence of fibrinogen, which is removed when plasma is allowed to clot.) FIG. 9 (Panel A) shows an expected thermogram (dashed line) obtained by calculating the weighted sum of the most abundant proteins (solid lines), minus HSA and fibrinogen. FIG. 9 (Panel B) shows the observed experimental thermogram for albumin-depleted serum. The agreement between the shape of the calculated and observed thermograms is excellent. Apart from confirming the major contribution by HSA to the peak at 62.8° C. in plasma thermograms, these data show that the contributions of other plasma proteins to thermograms can be amplified for more detailed study.

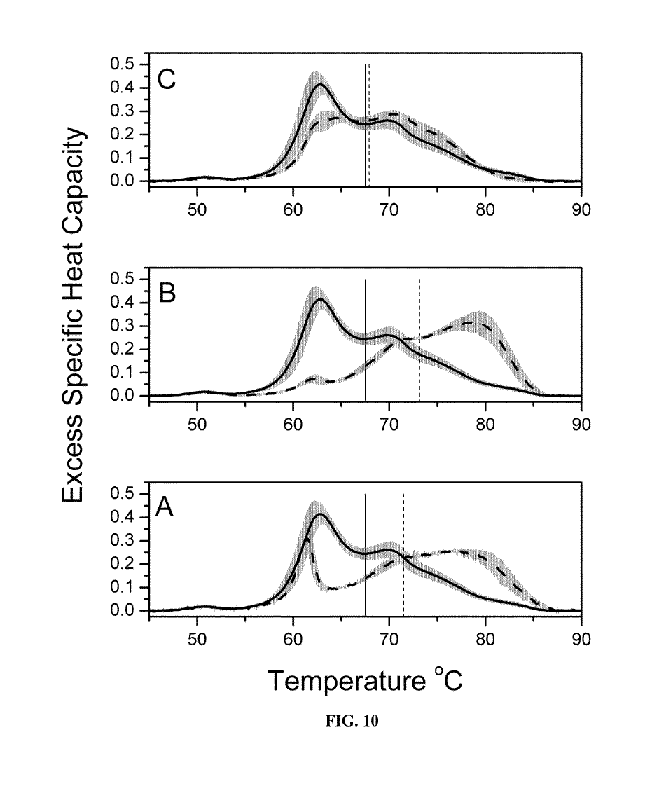

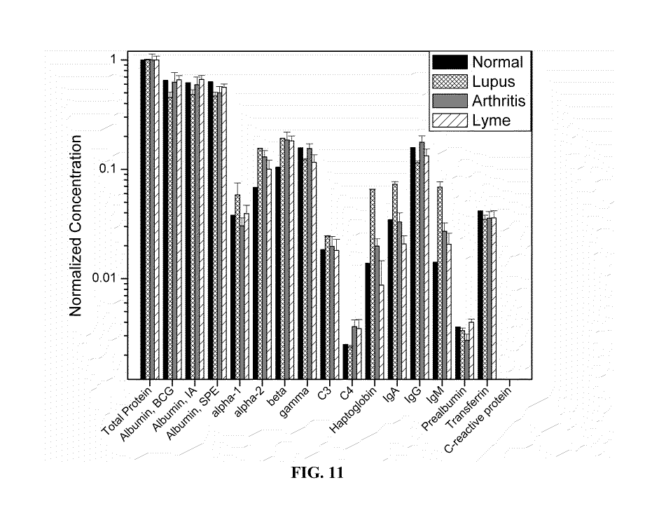

Distinctive thermograms for samples associated with a condition of interest. Plasma samples for subjects suffering from various conditions were obtained from BBI Diagnostics (West Bridgewater, Mass.). For comparison, plasma samples from 15 normal subjects were studied. Thermograms were obtained and compared as described herein, and the results are shown in FIG. 10. Shading indicates the standard deviation of the excess specific heat capacity at each temperature. The thermograms of diseased plasma (dashed lines) are distinctly different from thermograms obtained for plasma from normal subjects (solid lines). In addition, the thermograms for the diseased plasmas differ from one another, each showing distinctive patterns. FIG. 10 specifically compares average thermograms for subjects with three different conditions (rheumatoid arthritis, Lyme disease, systemic lupus) with the average normal thermogram. As noted, each disease appears to display a signature thermogram that differs from other diseases. In all cases, the 62.8° C. peak associated with HSA is greatly diminished, and the thermograms are shifted to higher temperatures. The solid vertical line is the first moment of the normal thermogram and the dashed vertical line is the first moment of the diseased thermogram.

FIG. 10 (Panel A) shows the thermogram for lupus. The first moment shifts from the normal value of 67.5 to 71.5° C. A sharp peak near 61° C. is evident that would be consistent with an elevation in haptoglobin concentration.

The thermogram for Lyme disease (FIG. 10 (Panel B)) is distinct from that seen for systemic lupus. The first moment at 73.15° C. is higher still, and the shape of the thermogram clearly differs from both normal and lupus thermograms.

FIG. 10 (Panel C) shows yet another distinctive thermogram for subjects suffering from rheumatoid arthritis. That thermogram is characterized by a first moment of 67.9° C., only slightly higher than normal, but with distinct changes in the shape relative to normal that are well beyond the standard deviations in the two thermograms. These collective results establish that embodiments of the methods of the presently-disclosed subject matter are useful and efficacious as clinical diagnostic tools. Thermograms can at a glance distinguish diseased states from normal, and have the potential for providing signatures for any specific condition of interest. The samples sizes used in these studies conform to the accepted standards for exploratory preclinical studies (Motulsky (1995)).

Origin of the altered thermograms. What causes the dramatic alterations in thermograms seen in FIG. 10? One possibility is that the concentrations of the major proteins in plasma are changed. This possibility was tested by experiments, and it was found that such is not the case. FIG. 11 shows the concentrations of the major plasma proteins for the same samples shown in FIG. 10. The data show that the protein composition of plasma from diseased subjects is in most cases indistinguishable from normal concentration values. Plasma from lupus patients represents a slight exception, with samples showing elevated concentrations of haptoglobin, IgA and IgM. Notably, albumin concentrations are normal for all of the diseased states, even though the thermogram peak at 62.8° C. that is characteristic of albumin is absent or greatly diminished in diseased samples (FIG. 10).

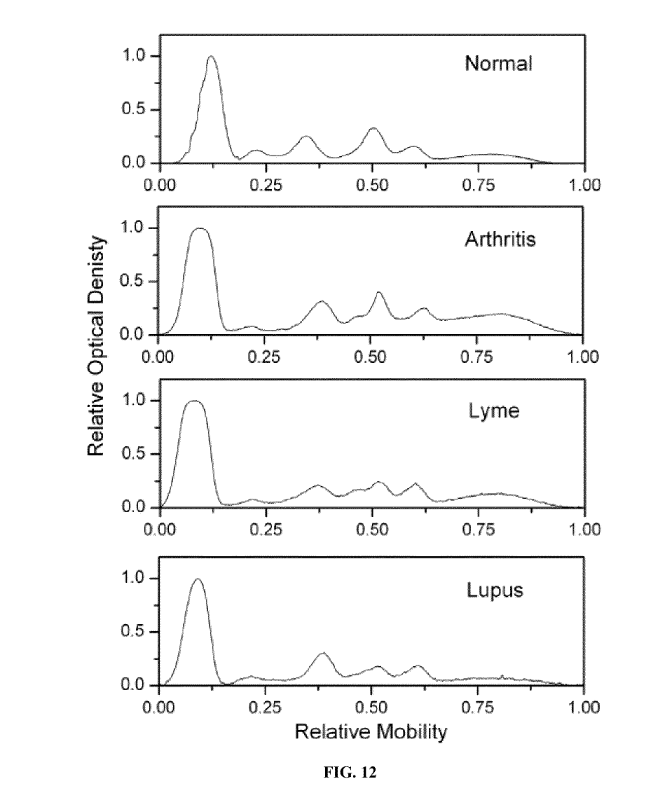

FIG. 12 shows protein electrophoresis patterns for normal plasma and the diseased states. Only subtle variations can be seen when comparing these traces, in contrast to the dramatic shifts in thermograms seen in FIG. 10. These data reveal a distinct advantage of the methods described herein. While whatever is present in plasma in the diseased state that differentiates samples from normal does not seem to drastically alter the concentrations or the sizes and charges of the plasma proteins (as revealed by electrophoresis), it does exert dramatic effects on the thermal properties of the proteins.

The most likely explanation for the shifts in the thermograms in FIG. 10 is that it results from binding interactions that involve the most abundant plasma proteins, particularly albumin. This view is consistent with the “interactome” hypothesis, that suggests that peptide and protein biomarkers specific for a particular disease are not free in plasma, but rather are bound to albumin or the immunoglobins. Such binding would result in thermal stabilization of the protein to which the biomarkers are bound, and a drastic alteration of the plasma thermogram with respect to normal. That is exactly what is seen in FIG. 10.

In order to test the hypothesis that shifted thermograms result from interactions, the following study was performed. Bromocresol green is a small organic molecule that binds to Site I of human serum albumin (HSA) with a binding constant of 7×10⁵ M⁻¹ (Peters (1996)). The consequences of such binding on plasma thermograms was studied by spiking a normal plasma sample with 30 micromolar bromocresol green. That concentration corresponds to roughly 1 equivalent of the compound per HSA protein molecule.

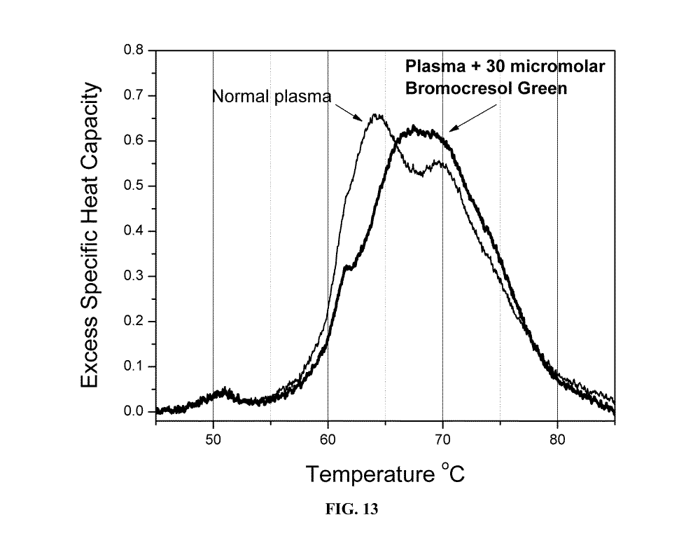

With reference to FIG. 13, the bromocresol green spike causes the plasma thermogram to shift to higher temperatures, in this case because the thermal denaturation of HSA is stabilized by binding of the small molecule. This test shows that addition of small components to plasma can in fact drastically alter the plasma thermogram, even though the actual melting of the added component can not itself be seen. The alteration results from stabilization of one or more of the more abundant components.

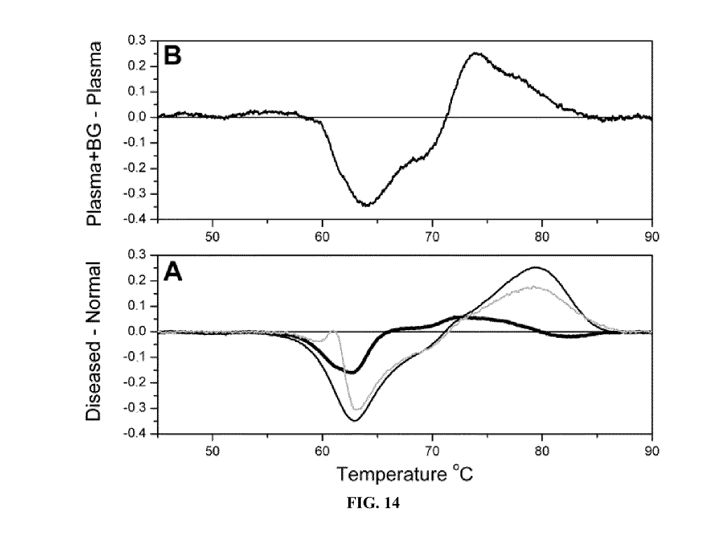

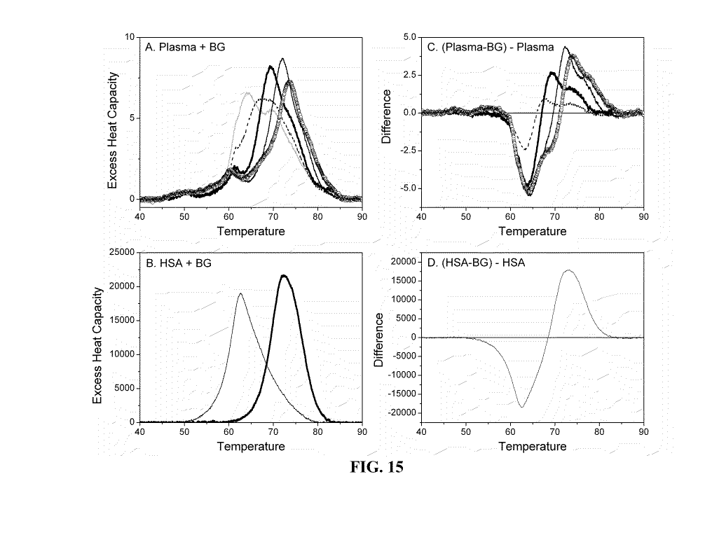

The results of another study are shown in FIG. 14, which indicate that the binding of bromocresol green to HSA within normal plasma mimic the effects of putative biomarker binding. FIG. 14 (Panel A) shows “difference thermograms” for diseased states, obtained by subtracting the normal thermogram from the diseased thermograms seen in FIG. 10. These difference plots feature a negative peak near 62° C., attributable to a shift in HSA denaturation to higher temperatures. Positive difference peaks are evident at 70° C. and higher, attributable to denaturation of ligated HSA (or other proteins). Such behavior can be mimicked by addition of bromocresol green (FIG. 14 (Panel B)). FIG. 14 (Panel B) shows a difference thermogram calculated from normal plasma samples with and without added bromocresol green. (More details of experiments showing the effects of bromocresol green on plasma and pure HSA are shown in FIG. 15). The shape of the difference thermogram is qualitatively similar to those seen for diseased plasma samples, suggesting that the “interactome” hypothesis has merit, and provides a plausible explanation for shifts in thermograms observed in FIG. 10.

The shifts in denaturation transition curves that accompany ligand binding to protein are well understood, and have been explained by a number of specific statistical mechanical and thermodynamic models (Brandts (1990) and Schellman (1958)). The effects of binding on the magnitude and exact shape of a melting transition curve depends precisely on the ligand binding affinity, enthalpy, and stoichiometry. Complex multiphasic transition curves can result from partial saturation. Peptide biomarkers in plasma could produce a myriad of thermogram shapes, depending on the exact proteins (and protein binding sites) that they occupy, and their affinity. The interactions of multiple unique biomarkers with different plasma proteins could produce unique, characteristic thermograms that reflect the underlying complexity of the interactions. While calorimetry may not sense signals arising from the denaturation of the biomarkers themselves, it is uniquely sensitive to interactions of these biomarkers with the more abundant plasma proteins.

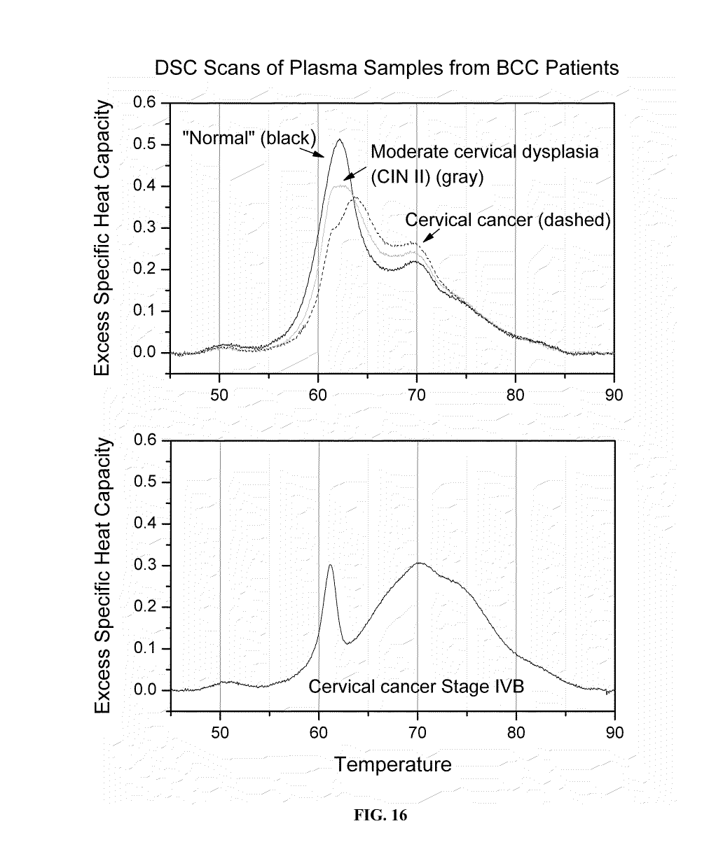

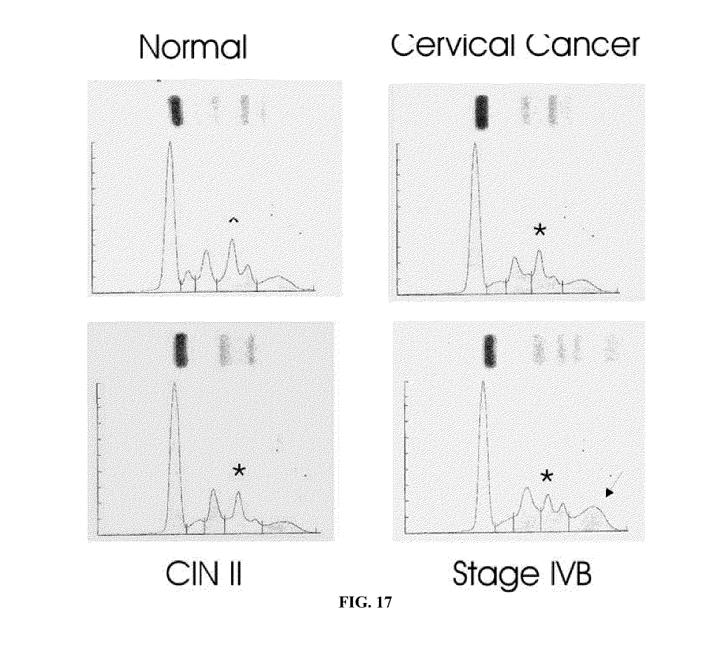

Distinctive thermograms for samples associated with additional conditions of interest. Plasma samples were obtained from subjects diagnosed with cervical cancer (samples obtained from a gynecological cancer tissue bank maintained at the University of Louisville). Thermograms were generated using the cervical cancer samples. The samples were associated with either moderate cervical dysplasia (CIN II), early stage cervical cancer, or stage IVB cervical cancer. With reference to FIG. 16, it was surprisingly found that unique thermograms are generated for particular stages of cervical cancer. As the condition progresses, the thermograms change. Compared to normal plasma, there are distinctive shifts in the thermograms as the disease progresses from moderate cervical dysplasia, through early stage cervical cancer, to the critically ill stage IVB cervical cancer. The changes in the thermograms are unique for each stage, and their patterns are further distinct in detail from the diseased states (lupus, Lyme disease, arthritis) shown in FIG. 10.

Aliquots of these identical samples were also analyzed by the FDA approved serum protein electrophoresis assay. Densitometric scans of the stained gels are shown for comparison in FIG. 17. In comparison to the DSC thermograms, these electrophoretic scans show only subtle changes throughout the progression of the cancer. Standard quantitative analysis of the electrophoresis did not reveal any dramatic systematic changes in the concentrations of protein fractions. This comparison indicates that thermograms reveal differences in plasma that are not readily visible by traditional serum plasma electrophoresis, indicating that the methods of the presently-disclosed subject matter are valuable complements to existing procedures.

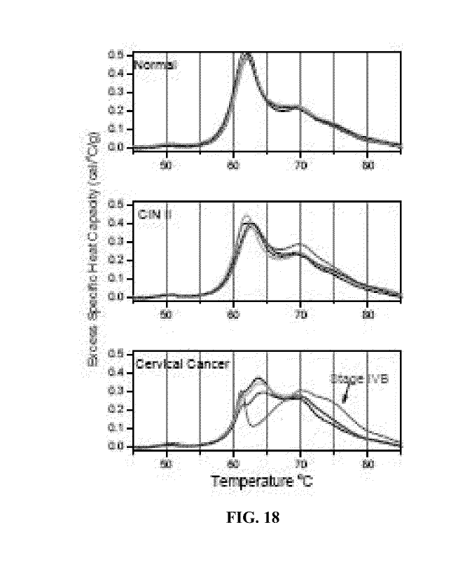

For the cervical cancers, thermograms were generated for several samples from the gynecological tissue bank. Samples from four normals, four CIN II cervical dysplasia, and four diagnosed cervical cancers were studied. These results are plotted in FIG. 18 and depict the reproducibility of the thermograms. The data for the diagnosed cervical cancers clearly show one pronounced outlier. These samples were originally ran blind, using deidentified samples without knowing the exact diagnoses. Upon identification of the outlier thermogram, it was subsequently identified as being from a stage IVB patient, late in progression, and clinically distinct from the other samples that had been provided. This provided an unexpected illustration of the present method's ability to distinguish between particular stages of the disease.

Using methods described herein, thermograms are obtained using plasma samples from normal subjects and from subjects diagnosed with a variety of cancers in order to explore and discover the range of patterns resulting from these diseases. Deidentified plasma samples are obtained from a tissue bank maintained at the University of Louisville. This resource maintains “discard” pieces of benign, premalignant, and malignant gynecological tissues for each patient donor, along with pre- and post-operative blood and urine samples, and ascites fluid (when possible). Plasma is prepared from blood samples by standard methods and was stored at −80° C.

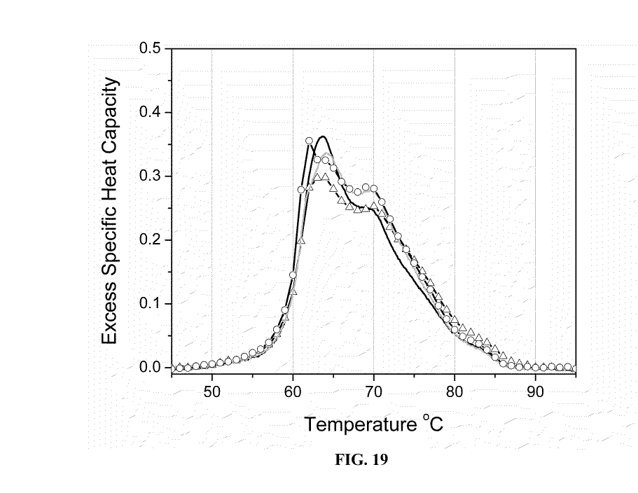

With reference to FIG. 19, thermograms were generated using samples from subjects diagnosed with ovarian cancer, endometrial cancer, and uterine cancer. The solid black line is the average thermogram from 10 normal female subjects; the open triangles show the average thermogram from 12 subjects with ovarian cancer; the solid gray line is the average thermogram from 8 subjects with endometrial cancer; the open circles show the average thermogram from 2 subjects with uterine cancer. These results indicate that ovarian cancer, endometrial cancer, and uterine cancer yield unique thermograms, that are distinct from normal thermograms, distinct from each other, and distinct from the thermograms associated with other conditions, e.g., cervical cancer, arthritis, lupus, Lyme disease.

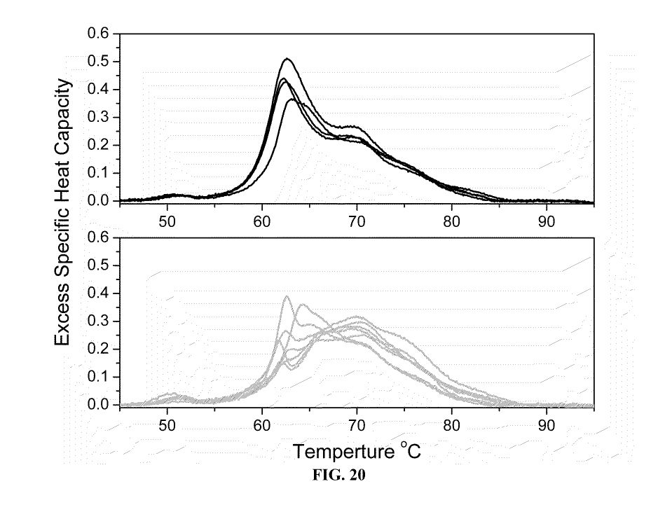

With reference to FIG. 20, thermograms were generated using samples from subjects diagnosed with melanoma. The solid black lines correspond to thermograms of samples obtained from subjects that have undergone successful treatment for melanoma and show no evidence of disease. The solid gray lines correspond to thermograms obtained from subjects with advanced melanoma. These results indicate that different stages of melanoma progression could yield unique thermograms. These results further indicate that melanoma thermograms are distinct from normal thermograms, and distinct from the thermograms associated with other conditions. These results further illustrate that the utility of embodiments of the method of the presently-disclosed subject matter for assessing or monitoring a treatment program, i.e., note the distinction between the thermograms associated with advanced melanoma, and the thermograms associated with successful treatment of melanoma, as well as the trend of the successful treatment thermograms towards a good simulation of a normal thermogram.

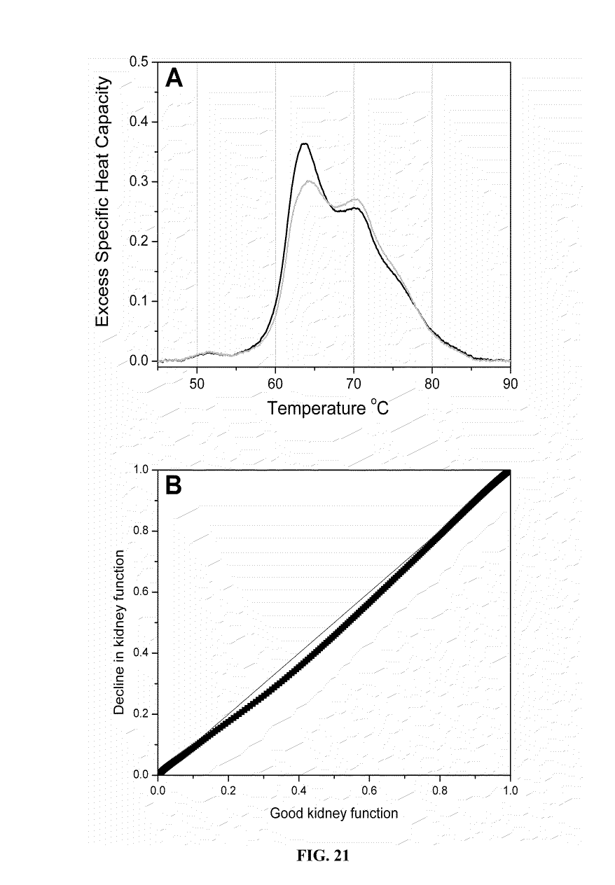

Using methods described herein, thermograms are obtained using plasma samples from normal subjects and from subjects diagnosed with a variety of conditions. With reference to FIG. 21, thermograms were generated using samples obtained prospectively from diabetic subjects exhibiting subsequent differences in future kidney function. Panel A shows average thermograms from two groups of subjects grouped on the basis of kidney function. The solid black line shows an average thermogram from 17 subjects with good kidney function, and the solid gray line is an average thermogram from 15 subjects exhibiting a decline in kidney function. Panel B shows a quantile-quantile plot. This is a graphical technique for determining if two data sets come from populations with a common distribution. If the two sets come from a population with the same distribution they will lie along the 45-degree reference line. The greater the departure from this reference line, the greater the evidence for the conclusion that the two data sets have come from populations with different distributions. Note the deviations from the 45-degree reference line. These results indicate that yet another condition-of-interest yields a unique thermogram.

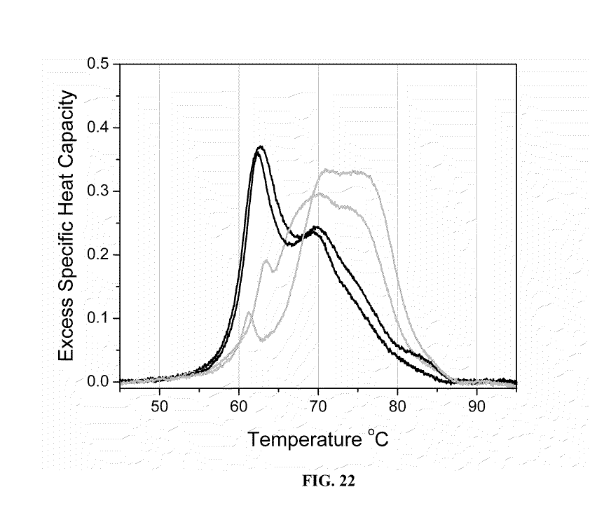

With reference to FIG. 22, thermograms were generated using samples from diabetic subjects with either minimal (CAD−) or severe (CAD+) coronary artery disease. The solid black lines correspond to CAD− patients and the solid gray lines to CAD+ patients. These results provide further evidence that each unique condition can yield a unique thermogram, useful for the methods of the presently-disclosed subject matter.

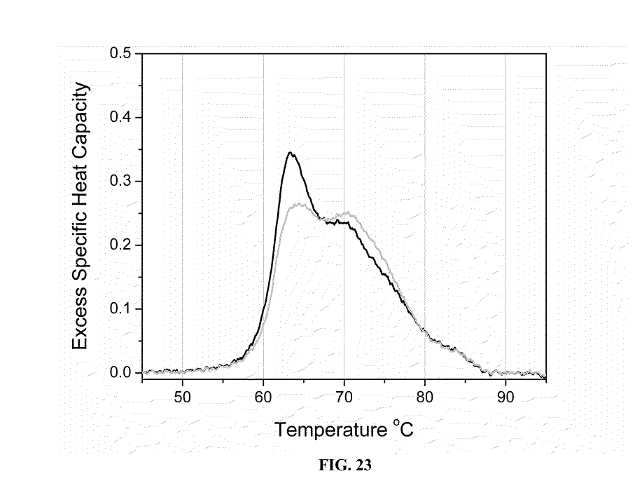

With reference to FIG. 23, thermograms were generated using samples from subjects with amyotrophic lateral sclerosis (ALS). The solid black line corresponds to the average thermogram obtained from 9 normal subjects; the solid gray line corresponds to the average thermogram obtained from 9 subjects with ALS disease. These results provide still further evidence that each unique condition can yield a unique thermogram, useful for the methods of the presently-disclosed subject matter.

The results of the studies described herein indicate that the methods of the presently-disclosed subject matter are extremely sensitive to binding interactions between proteins. Changes in low-abundance “biomarkers” of conditions of interest that cannot be detected by known methods such as mass spectroscopy or 2-dimensional electrophoresis can be detected with sensitivity using the methods of the presently-disclosed subject matter.

The methods of the presently-disclosed subject are sensitive not only to changes in protein compositions in a noninteracting mixture, but also to interactions resulting from increased concentrations of smaller components (e.g., “biomarkers”) that would themselves not be directly observed. In either case, reproducible signature changes in thermograms relative to normal samples are seen.

Normal thermograms and thermograms for specific conditions of interest are reproducible and distinct. A thermogram for a specific condition of interest is different than a normal thermogram, and is also different than thermograms for other conditions of interest, i.e., they are poor simulations of one another. Each condition of interest has a distinctive and characteristic thermogram. Indeed, in some embodiments, different stages of a condition of interest have distinctive and characteristic thermograms. Therefore, the methods of the presently-disclosed subject matter have beneficial clinical utility and research utility. Benefits of the methods include, the sensitivity, simplicity, non-invasive sample collection, ability to work with low-volume samples, ease of sample preparation, and the capacity for high-throughput.

Materials and Methods

Pure protein samples. Human serum albumin (HSA) (lot # 113K7601), immunoglobulin G (IGG) (lot # 415781/1), immunoglobulin A (IGA) (lot # 105K3777), α1-acid glycoprotein (AAG) (lot # 073K7607), α1-antitrypsin (AAT) (lot # 033K7603), fibrinogen (FIB) (lot # 083K7604), transferrin (TRF) (lot # 123K14511), haptoglobin (HPT) (lot # 055K1664) and immunoglobulin M (IGM) (lot # 016K4876) were purchased from Sigma-Aldrich Chemical Co. (St. Louis, Mo.). α1-Antichymotrypsin (ACT) (lot # B58700), complement C3 (C3) (lot # D33204), complement C4 (C4) (lot # D34721), ceruloplasmin (CER) (lot # B70322), α2-macroglobulin (A2M) (lot # B73605) and prealbumin (PRE) (lot # B68296) were purchased from Calbiochem. C-reactive protein (CRP) (lot # 32F0305FP) was purchased from Life Diagnostics.

Manufactured mixtures. By using available purified plasma proteins, solution mixtures of any desired composition can be made and thermograms for these preparations can be obtained. Such is done, in order to match experimental thermograms of normal and diseased plasma/serum samples. This approach allows for an exploration of the effects of individual components on thermogram shape.

Standard reference serum. A serum reference material (sample # 16910) was purchased from Sigma-Aldrich Chemical Co. (St. Louis, Mo.). A standardized human serum sample can be provided with a certificate of analysis that includes certified values for the concentrations (g/L) of the 15 most abundant proteins, along with the uncertainty in the concentration determination. Concentrations of each sample are determined on the same sample independently by multiple different laboratories. Each sample is provided as a lyophilized portion under nitrogen, and a strict standardized protocol for reconstitution of the material is provided. Thermograms obtained for such materials are useful for multicomponent analysis, since the protein concentrations that are being sought by the numerical analyses procedure are precisely known for the experimental sample. The goodness of fits can thus be rigorously evaluated.

Plasma samples. Normal plasma samples (lot # JA053759, JA053761, JA053763, JA053764, JA053765, JA053766, JC014372, JM034968, JM034969, JM034970, JM034971) were purchased from Innovative Research (Southfield, Mich.) and were also obtained from the Gynecological Cancer Repository of the James Graham Brown Cancer Center. Plasma from subjects suffering from Lyme disease (lot # BM146897, BM140032, BM140031, BM140028), systemic lupus erythematosis (lot # BM142168, BM142160) and rheumatoid arthritis (lot # BM204810, BM205222, BM203373, BM202803, BM200182) were purchased from BBI Diagnostics (West Bridgewater, Mass.).

Sample preparation. IGM, C3, C4 and CRP were purchased as solutions in buffer, lyophilized to dryness and then re-constituted in a smaller volume of ultrapure water (18.2 MS2-cm) to yield a concentration suitable for DSC. PRE, A2M, CER, ACT were purchased as a powder lyophilized from buffer and were reconstituted with ultrapure water. HSA, IGG, IGA, AAG, AAT, FIB, TRF and HPT were reconstituted with 10 mM potassium phosphate, 150 mM NaCl, pH 7.5. Reference serum was reconstituted according to the guidelines. Pure proteins and reference serum were dialyzed for 24 h at 4° C. against 10 mM potassium phosphate, 150 mM NaCl, pH 7.5 to ensure complete solvent exchange. Pure proteins were diluted with dialysate to a concentration suitable for DSC. Reference serum was diluted 25-fold with the dialysate. Plasma samples (100 μL) were dialyzed for 24 h at 4° C. against 10 mM potassium phosphate, 150 mM NaCl, 0.38% (w/v) sodium citrate, pH 7.5 to ensure complete solvent exchange then diluted 25-fold with the same buffer. All samples (0.45 micron, cellulose acetate or polyethersulfone) and buffers (0.22 micron, polyethersulfone) were filtered before use. Pure protein concentrations were quantitated spectrophotometrically using the following extinction coefficients (ε280; L-1·g-1·cm-1): HSA, 0.53; IGG, 1.38; IGA, 1.32; AAG, 0.89; AAT, 0.53; FIB, 1.55; TRF, 1.12; HPT, 1.2; IGM, 1.18; ACT, 0.62; C3, 0.97; C4, 0.92; CER, 1.49; A2M, 0.893; PRE, 1.41; CRP, 1.95.

DSC protocol. An automated capillary Differential Scanning Calorimeter (DSC) (MicroCal, LLC, Northampton, Mass.) was used for the studies described herein. Samples and dialysate were stored in 96-well plates at 5° C. until being loaded into the calorimeter using the robotic attachment. Scans were recorded from 20-110° C. at 1° C./min using the mid feedback mode, a filtering period of 2 s and with a pre-scan thermostat of 15 min. Data were analyzed using Origin 7.0. Sample scans were first corrected for the instrument baseline by subtracting an appropriate buffer scan. Nonzero baselines were then corrected by applying a linear baseline fit. Scans were finally normalized for the gram concentration of protein. For the pure protein samples, protein concentrations were determined spectrophotometrically as outlined herein. Total protein concentrations of the reference serum and plasma samples were measured by the bicinchoninic acid method (Pierce, Rockford, Ill.). Thermograms were plotted as Excess Specific Heat Capacity (cal/° C.g) versus temperature.

Clinical Laboratory Testing. Both total protein and the concentration of the individual major serum proteins are measured, for example, immunoglobulins G, A and M, transferrin, haptoglobin, prealbumin, complement factors C3 and C4, ceruloplasmin, apolipoproteins A1 and B, α1-antitrypsin, α1-acid glycoprotein, and C-reactive protein. In addition, serum (or plasma) protein electrophoresis is performed on each sample. All of these assays are performed by FDA approved, standard clinical laboratory procedures. The concentrations of the specific serum proteins and the SPE patterns are correlated with the thermograms determined by the methods described herein.

Lipoproteins (HDL, LDL, VLDL, and chylomicrons) are more complex than the other serum proteins. They contain not only the apolipoproteins, but also cholesterol and triglyceride, as well as other minor components. The lipoproteins are likely to cause a significant signal in the thermogram patterns. Therefore, cholesterol and triglyceride of the samples are also measured. Cholesterol and triglyceride is measured on the Vitros by enzymatic methods.

C-reactive protein (CRP) is normally present at a low concentration, which is unlikely to contribute to the thermogram pattern. However, during the acute phase reaction, which is common among sick patients, the concentration of CRP can be high enough to be detectable by the methods described herein.

Clinical assay methods. Protein electrophoresis was performed on agarose gels using the SPIFE 3000 and scanned with the QUICKSCAN 2000 (Helena Laboratories, Beaumont, Tex.). Total protein was measured by the biuret method on the Ortho Vitros 950 (Vitros) (Ortho-Clinical Diagnostic, Rochester, N.Y.) chemistry analyzer. Albumin was measured on the Vitros by the bromocresol green dye binding assay or by an immunoturbidometric assay on the Cobas Integra 800 (Integra) (Roche, Indianapolis, Ind.). Albumin concentrations were also determined from the fraction percent on the protein electrophoresis assay along with the total protein concentration. Specific serum proteins (IGG, IGA, TRF, HPT, IGM, C3, C4, PRE, CRP) were measured by immunoturbidimetry on the Integra.

Column depletion experiments. Reference serum was depleted of HSA using the SwellGel Blue™ albumin removal kit with some minor modifications to the manufacturer's protocol (Pierce, Rockford, Ill.). The serum sample was diluted 10-fold into 10 mM potassium phosphate, pH 7.5 in order to achieve salt conditions and albumin concentrations required for good column binding. Diluted serum (200 μL) was applied to a column containing 2 SwellGel™ discs. An HSA-depleted fraction was obtained following the standard protocol. A single 200 μL volume of the supplied binding/wash buffer was used to obtain a wash fraction. Finally, an eluted HSA fraction was obtained from a single 200 μL addition of the supplied elution buffer. In order to obtain a greater volume of each fraction for subsequent experiments, multiple columns were run using an identical protocol and each of the fractions pooled. Fractions for DSC analysis were dialyzed for 24 h at 4° C. against 10 mM potassium phosphate, 150 mM NaCl, pH 7.5 and diluted as necessary with dialysate. DSC scans were performed on an N-DSC II instrument (Calorimetry Sciences Corporation, Provo, Utah) from 20-110° C. at 1° C./min with a pre-scan equilibration time of 10 min. Data were analyzed using Origin 7.0.

Fractionation and Generation of Thermograms from Sample Fractions.

Plasma from a healthy normal subject was fractionated using Suprdex 75 (GE Healthcare). Fractions were taken at different points along the elution profile and subjected to DSC. The thermograms of each fraction (FIG. 5) reveal the major protein components, with reference to the thermograms of purified plasma proteins (FIG. 7). The combined data yield a multidimensional view of plasma protein composition, with temperature on one axis, elution time on another, and excess heat capacity on the third. For conditions of interest, the thermograms of particular fractions are expected to be altered, while others will be unchanged.

Methods as disclosed herein, including fractionation and generation of thermograms, provide a novel multidimensional signature of a sample, and allows identification of the component most responsible for shifts in the thermogram for plasma, e.g., as in a subject having a condition of interest.

With reference to FIG. 5, the left panel shows the elution profile obtained for fractionation of healthy normal plasma using Superdex 75 gel filtration media. Fractions along the elution profile were subjected to differential scanning calorimetry, with the results show in the right panel. Each trace is data from a particular fraction along the elution profile. In gel filtration chromatography, higher molecular weight protein elute first from the column. The peaks in each colored trace in the right panel correspond to different size fractioned proteins (see FIG. 7 for thermograms of pure plasma proteins).

Protocol. Undiluted plasma or serum (300-500 μL) were fractionated using gel filtration chromatography (Superdex 75 10/300 GL or Superdex 200 10/300 GL; manufacturers instructions and Superdex-related materials are incorporated herein by this reference). The columns were prepared for use following standard procedures and finally rinsed with a standard phosphate buffer (for plasma: 10 mM potassium phosphate, 150 mM sodium chloride, 0.38% sodium citrate, pH 7.5; for serum: 10 mM potassium phosphate, 150 mM sodium chloride, 0.38% sodium citrate, pH 7.5) before use. Fractions (50-200 μL) were collected overnight at a flow rate of 0.05 to 0.2 mL/min. The total protein concentration of each fraction was estimated spectrophotometrically. Fractions were selected for DSC analysis based on the elution profile and the protein concentration. Based on the estimated protein concentration and the volume requirements for DSC analysis, the selected fractions were diluted (with gel filtration running buffer) or pooled as necessary before loading into 96 well plates for DSC analysis. A final total protein concentration was determined colorimetrically for each DSC sample (BCA assay; Pierce Biotechnology Inc., Rockford, Ill.) in a microplate format (Tecan U.S., Research Triangle Park, N.C.). The colorimetric total protein concentrations were subsequently used to normalize the DSC thermograms. DSC thermograms were collected and analyzed according to the DSC protocol using the gel filtration running buffer as the reference buffer.

Throughout this document, various references are mentioned. All such references are incorporated herein by reference, including the references set forth in the following list:
1. Anderson, N. L. and N. G. Anderson, The human plasma proteome: history, character, and diagnostic prospects. Mol Cell Proteomics, 2002. 1(11): p. 845-67.
2. Anderson, N. L., M. Polanski, R. Pieper, T. Gatlin, R. S. Tirumalai, T. P. Conrads, T. D. Veenstra, J. N. Adkins, J. G. Pounds, R. Fagan, and A. Lobley, The human plasma proteome: a nonredundant list developed by combination of four separate sources. Mol Cell Proteomics, 2004. 3(4): p. 311-26.
3. Omenn, G. S., D. J. States, M. Adamski, T. W. Blackwell, R. Menon, H. Hermjakob, R. Apweiler, B. B. Haab, R. J. Simpson, J. S. Eddes, E. A. Kapp, R. L. Moritz, D. W. Chan, A. J. Rai, A. Admon, R. Aebersold, J. Eng, W. S. Hancock, S. A. Hefta, H. Meyer, Y. K. Paik, J. S. Yoo, P. Ping, J. Pounds, J. Adkins, X. Qian, R. Wang, V. Wasinger, C. Y. Wu, X. Zhao, R. Zeng, A. Archakov, A. Tsugita, I. Beer, A. Pandey, M. Pisano, P. Andrews, H. Tammen, D. W. Speicher, and S. M. Hanash, Overview of the HUPO Plasma Proteome Project: results from the pilot phase with 35 collaborating laboratories and multiple analytical groups, generating a core dataset of 3020 proteins and a publicly-available database. Proteomics, 2005. 5(13): p. 3226-45.
4. Aebersold, R., L. Anderson, R. Caprioli, B. Druker, L. Hartwell, and R. Smith, Perspective: a program to improve protein biomarker discovery for cancer. J Proteome Res, 2005. 4(4): p. 1104-9.
5. Anderson, N. L., The roles of multiple proteomic platforms in a pipeline for new diagnostics. Mol Cell Proteomics, 2005. 4(10): p. 1441-4.
6. Ebert, M. P., M. Korc, P. Malfertheiner, and C. Rocken, Advances, challenges, and limitations in serumproteome-based cancer diagnosis. J Proteome Res, 2006. 5(1): p. 19-25.
7. Mor, G., I. Visintin, Y. Lai, H. Zhao, P. Schwartz, T. Rutherford, L. Yue, P. Bray-Ward, and D. C. Ward, Serum protein markers for early detection of ovarian cancer. Proc Natl Acad Sci USA, 2005. 102(21): p. 7677-82.
8. Rosenblatt, K. P., P. Bryant-Greenwood, J. K. Killian, A. Mehta, D. Geho, V. Espina, E. F. Petricoin, 3rd, and L. A. Liotta, Serum proteomics in cancer diagnosis and management. Annu Rev Med, 2004. 55: p. 97-112.
9. Wulfkuhle, J. D., L. A. Liotta, and E. F. Petricoin, Proteomic applications for the early detection of cancer. Nat Rev Cancer, 2003. 3(4): p. 267-75.
10. Wulfkuhle, J. D., C. P. Paweletz, P. S. Steeg, E. F. Petricoin, 3rd, and L. Liotta, Proteomic approaches to the diagnosis, treatment, and monitoring of cancer. Adv Exp Med Biol, 2003. 532: p. 59-68.
11. O'Connell, T. X., T. J. Horita, and B. Kasravi, Understanding and interpreting serum protein electrophoresis. Am Fam Physician, 2005. 71(1): p. 105-12.
12. Craig, W., T. Ledue, and R. Ritchie, Plasma Proteins: Clinical Utility and Interpretation. 2004, Scarborough, Me.: Foundation for Blood Research. 152.
13. Anderson, L. and N. G. Anderson, High resolution two-dimensional electrophoresis of human plasma proteins. Proc Natl Acad Sci USA, 1977. 74(12): p. 5421-5.
14. Anderson, N. L. and N. G. Anderson, A two-dimensional gel database of human plasma proteins. Electrophoresis, 1991. 12(11): p. 883-906.
15. Gygi, S. P. and R. Aebersold, Mass spectrometry and proteomics. Curr Opin Chem Biol, 2000. 4(5): p. 489-94.
16. Liotta, L. A., E. C. Kohn, and E. F. Petricoin, Clinical proteomics: personalized molecular medicine. JAMA, 2001. 286(18): p. 2211-4.
17. Yates, J. R., 3rd, Mass spectrometry. From genomics to proteomics. Trends Genet, 2000. 16(1): p. 5-8.
18. Adkins, J. N., S. M. Varnum, K. J. Auberry, R. J. Moore, N. H. Angell, R. D. Smith, D. L. Springer, and J. G. Pounds, Toward a human blood serum proteome: analysis by multidimensional separation coupled with mass spectrometry. Mol Cell Proteomics, 2002. 1(12): p. 947-55.
19. Liotta, L. A. and E. F. Petricoin, Serum peptidome for cancer detection: spinning biologic trash into diagnostic gold. J Clin Invest, 2006. 116(1): p. 26-30.
20. Liotta, L. A., M. Ferrari, and E. Petricoin, Clinical proteomics: written in blood. Nature, 2003. 425(6961): p. 905.
21. Zhou, M., D. A. Lucas, K. C. Chan, H. J. Issaq, E. F. Petricoin, 3rd, L. A. Liotta, T. D. Veenstra, and T. P. Conrads, An investigation into the human serum “interactome”. Electrophoresis, 2004. 25(9): p. 1289-98.
22. Fenby, D. V., Heat: its measurement from Balileo to Lavosier. Pure Applied Chemistry. 1987. 59: p. 91-1000.
23. Biltonen, R. L. and E. Freire, Thermodynamic characterization of conformational states of biological macromolecules using differential scanning calorimetry. CRC Crit Rev Biochem, 1978. 5(2): p. 85-124.
24. Brandts, J. F. and L. N. Lin, Study of strong to ultratight protein interactions using differential scanning calorimetry. Biochemistry, 1990. 29(29): p. 6927-40.
25. Bruylants, G., J. Wouters, and C. Michaux, Differential scanning calorimetry in life science: thermodynamics, stability, molecular recognition and application in drug design. Curr Med Chem, 2005. 12(17): p. 2011-20.
26. Freire, E., Differential scanning calorimetry. Methods Mol Biol, 1995. 40: p. 191-218.
27. Sanchez-Ruiz, J. M., Differential scanning calorimetry of proteins. Subcell Biochem, 1995. 24: p. 133-76.
28. Schellman, J. A., The factors affecting the stability of hydrogen-bonded polypeptide structures in solution. Journal of Physical Chemistry, 1958. 62: p. 1485-1494.
29. Peters, T., Jr., All About Albumin: Biochemistry, Genetics and Medical Applications. 1996, San Diego Academic Press.
30. Doumas, B. T., D. D. Bayse, R. J. Carter, T. Peters, Jr., and R. Schaffer, A candidate reference method for determination of total protein in serum. I. Development and validation. Clin Chem, 1981. 27(10): p. 1642-50.
31. Corcoran, R. M. and S. M. Durnan, Albumin determination by a modified bromcresol green method. Clin Chem, 1977. 23(4): p. 765-6.
32. Allain, C. C., L. S. Poon, C. S. Chan, W. Richmond, and P. C. Fu, Enzymatic determination of total serum cholesterol. Clin Chem, 1974. 20(4): p. 470-5.
33. Cooper, G. R., G. L. Myers, S. J. Smith, and E. J. Sampson, Standardization of lipid, lipoprotein, and apolipoprotein measurements. Clin Chem, 1988. 34(8B): p. B95-105.
34. Eda, S., J. Kaufmann, W. Roos, and S. Pohl, Development of a new microparticle-enhanced turbidimetric assay for C-reactive protein with superior features in analytical sensitivity and dynamic range. J Clin Lab Anal, 1998. 12(3): p. 137-44.
35. Gorman, L. S. and D. Adkins, Apoprotein assays: radial immunodiffusion versus an immunoturbidometric technique. Clin Lab Sci, 1991. 4(2): p. 110-2.
36. Larkin, T. C., T. A. Fingleton, M. McCusker, M. Cassidy, G. Gunzer, C. A. Lepp, and E. Gamboa, A new Olympus assay for the determination of ceruloplasmin. Clin Lab, 2004. 50(3-4): p. 193-203.
37. Levinson, S. S. and S. G. Wagner, Immunonephelometric/turbidimetric apolipoprotein B assays for the clinical laboratory. Clin Chim Acta, 1993. 223(1-2): p. 31-42.
38. Lolekha, P. H., A. Chittamma, W. L. Roberts, P. Sritara, S. Cheepudomwit, and P. Suriyawongpaisal, Comparative study of two automated high-sensitivity C-reactive protein methods in a large population. Clin Biochem, 2005. 38(1): p. 31-5.
39. Molinari, E. A., P. F. Pichler, H. Grillhofer, and G. M. Kostner, Immunoturbidimetric determination of lipoprotein(a): improvement in the measurement of turbid and triglyceride-rich samples. Clin Chim Acta, 1995. 235(1): p. 59-69.
40. Sakurabayashi, I., Y. Saito, T. Kita, Y. Matsuzawa, and Y. Goto, Reference intervals for serum apolipoproteins A-I, A-II, B, C-II, C-III, and E in healthy Japanese determined with a commercial immunoturbidimetric assay and effects of sex, age, smoking, drinking, and Lp(a) level. Clin Chim Acta, 2001. 312(1-2): p. 87-95.
41. Sanchez, A., J. L. Mirabel, E. Barrenechea, J. Eugui, A. Puelles, and A. Castaneda, Evaluation of an improved immunoturbidimetic assay for serum C-reactive protein on a COBAS INTEGRA 400 Analyzer. Clin Lab, 2002. 48(5-6): p. 313-7.
42. Howerton, D. A., I. J. Check, and R. L. Hunter, Densitometric quantitation of high resolution agarose gel protein electrophoresis. Am J Clin Pathol, 1986. 85(2): p. 213-8.
43. Cantor, C. and P. Schimmel, Biophysical Chemistry Part 1: The conformation of biological macromolecules. 1980, San Francisco: W.H. Freeman. page 37.
44. Lodder, R. A. and Hieftje, G. M., Quantile analysis: a method for characterizing data distributions. Applied Spectroscopy, 1988. 42: 1512-1520.
45. Young, I. T., Proof without prejudice: use of the Kolmogorov-Smirnov test for the analysis of histograms from flow systems and other sources. Journal of Histochemistry and Cytochemistry, 1977. 25: 935-941.
46. Motulsky, H. Intuitive Biostatisitics, (Oxford University Press, New York, 1995).
47. Garbett, N. C., Miller, J. J., Jenson, A. B., Miller, D. M., Chaires, J. B., Interrogation of the Plasma Proteome with Differential Scanning Calorimetry. Clinical Chemistry, 2007. 53: 2012-2014.
48. Garbett, N. C., Miller, J. J., Jenson, A. B., Chaires, J. B., Calorimetric Analyisis of the Plasma Proteome. Seminars in Nephrology 27(6): 621-6.
49. Garbett, N. C., Miller, J. J., Jenson, A. B., Chaires, J. B., Ligand Binding Alters the Calorimetric Thermogram of Albumin. J. Clinical Ligand Assay, 2006*. 29:4, 1-4.(*Article written and submitted in 2007, notwithstanding Winter 2006 identification of J. of Clinical Ligand Assay publication).
50. Garbett, N. C., Miller, J. J., Jenson, A. B., Chaires, J. B. (2008) Calorimetry outside the box: a new window into the plasma proteome. Biophysical Journal 94: 1377-1383.
51. Petricoin, E. F., C. Belluco, R. P. Araujo, and L. A. Liotta (2006) The blood peptidome: a higher dimension of information content for cancer biomarker discovery. Nat Rev Cancer 6:961-967.
52. Wall, M. E., A. Rechtsteiner, and L. M. Rocha, Singular value decomposition and principal component analysis, in A practical approach to microarray data analysis, D. P. Berrar, W. Dubitzky, and M. Granzow, Editors. 2002, Springe: Berlin. p. 91-109.
53. Cattell, R. B., The Scree test for the number of factors. Multivariate Behavioral Research, 1966. 1: p. 245-276.
54. Loder, R. A. and G. M. Hieftje, Quantile analysis: a method for characterizing data distributions. Applied Spectroscopy 1988. 42: p. 1512-1520.

What is claimed is:

1. A method of identifying biomarkers useful for diagnosing a condition of interest in a subject, comprising:
providing a test sample associated with the condition of interest;
fractionating the test sample to obtain fractions of the test sample;
generating a signature thermogram for at least one fraction of the test sample;
comparing the signature thermogram to a sibling standard thermogram; and
determining whether the signature thermogram is a good simulation or a poor simulation of the sibling standard thermogram.

2. The method of claim 1, wherein the sibling standard thermogram is a sibling positive standard thermogram generated using a positive control sample including a candidate biomarker.

3. The method of claim 2, wherein the candidate biomarker is selected from a protein, a nucleic acid, a phospholipid, and a small organic molecule.

4. The method of claim 2, wherein the candidate biomarker is identified as an actual biomarker when the signature thermogram of a fraction of the test sample is a good simulation of sibling positive standard thermogram of the candidate biomarker.

5. The method of claim 1, and further comprising:
providing a negative control sample associated with an absence of the condition of interest;
fractionating the control sample to obtain sibling fractions of the control sample; wherein the sibling standard thermogram is a sibling negative standard thermogram generated for a sibling fraction of the negative control sample; and
identifying a fraction of the test sample having a unique component relative to the sibling fraction of the negative control sample due to the signature thermogram being a poor simulation of the sibling negative standard thermogram.

6. The method of claim 5, and further comprising:
testing the fraction of the test sample having a unique component to determine the identity of the unique component; and
classifying the identified unique component as a biomarker useful for diagnosing the condition of interest.

7. The method of claim 1, wherein the fractionating is conducted using gel filtration, gel electrophoresis, chromatographic fractionation, separation columns, immunoaffinity, centrifugation, mass spectroscopy, bioinformatic fractionation, or combinations thereof.

8. The method of claim 1, wherein the fractionation results in separation by size, mass, shape, charge, or thermal stability the sample components.

9. The method of claim 1, wherein the condition of interest is selected from the group consisting of: a cancer, an autoimmune disease, and a microbial infection.

10. The method of claim 9, wherein the condition is selected from the group consisting of: brain cancer, central nervous system (CNS) cancer, cervical cancer, endometrial cancer, lung cancer, leukemia, lymphoma, melanoma, multiple myeloma, ovarian cancer, vulvar cancer, a cancer of glial cells, including astrocytes, oligodendrocytes, ependymal cells; a cancer of neurons; a cancer of lymphatic tissue; a cancer of blood vessels; a cancer of cranial nerves; a cancer of the brain envelope; a cancer of the pitutitary gland; a cancer of the pineal gland; a metastatic cancer of the brain, a secondary cancer, wherein the primary cancer is a brain cancer, grade 1 astrocytoma, grade 2 astrocytoma, grade 3 astrocytoma, glyoblastoma mutiforme, moderate cervical dysplasia (CIN II), early stage cervical cancer, stage IVB cervical cancer, rheumatoid arthritis, multiple sclerosis, systemic lupus, Lyme disease, Dengue fever, hepatitis, amyotrophic lateral sclerosis (ALS), anemia, cardiac disease, diabetes, and renal disease.

11. The method of claims 1, wherein the wherein the samples are selected from: plasma sample, serum sample, a blood sample, an ascites fluid sample, a cerebral spinal fluid sample, a peritoneal fluid sample, a saliva sample, a senovial fluid sample, an ocular fluid sample, and a urine sample.

12. A method of diagnosing or monitoring a condition of interest in a subject, comprising:
providing a test sample to a subject;
fractionating the test sample to obtain fractions of the test sample;
generating a signature thermogram for each fraction of the test sample;
comparing a signature thermogram with a sibling standard thermogram and/or a sibling signature thermogram; and
identifying a status of the subject;
wherein the standard thermogram is selected from a positive standard thermogram associated with a presence of the condition of interest, and a negative standard thermogram associated with an absence of the condition of interest.

13. The method of claim 12, further comprising providing multiple standard thermograms associated with different conditions of interest or different stages of a condition of interest.

14. The method of claim 12, further comprising:
identifying the status of the subject as having the condition of interest when
the signature thermogram of a fraction of the test sample is a poor simulation of the negative standard thermogram; and/or
the signature thermogram of a fraction of the test sample is a good simulation of the positive standard thermogram; and
identifying the status of the subject as lacking the condition of interest when
the signature thermogram of a fraction of the test sample is a good simulation of the negative standard thermogram; and/or
the signature thermogram of a fraction of the test sample is a poor simulation of the positive standard thermogram.

15. The method of claim 12, further comprising:
providing a control sample;
fractionating the control sample to obtain sibiling fractions of the control sample;
generating a sibling standard thermogram for the sibling fractions of the control sample;
comparing the signature thermogram with the sibling standard thermogram; wherein the control sample is selected from a positive control sample, wherein a series of sibling positive standard thermograms are generated; and a negative control sample, wherein a series of sibling negative standard thermograms are generated.

16. The method of claim 12, wherein the fractionating is conducted using gel filtration, gel electrophoresis, chromatographic fractionation, separation columns, immunoaffinity, centrifugation, mass spectroscopy, bioinformatic fractionation, or combinations thereof.

17. The method of claim 12, wherein the fractionation results in separation by size, mass, shape, charge, or thermal stability the sample components.

18. The method of claim 12, wherein the condition of interest is selected from the group consisting of: a cancer, an autoimmune disease, and a microbial infection.

19. The method of claim 18, wherein the condition is selected from the group consisting of: brain cancer, central nervous system (CNS) cancer, cervical cancer, endometrial cancer, lung cancer, leukemia, lymphoma, melanoma, multiple myeloma, ovarian cancer, vulvar cancer, a cancer of glial cells, including astrocytes, oligodendrocytes, ependymal cells; a cancer of neurons; a cancer of lymphatic tissue; a cancer of blood vessels; a cancer of cranial nerves; a cancer of the brain envelope; a cancer of the pitutitary gland; a cancer of the pineal gland; a metastatic cancer of the brain, a secondary cancer, wherein the primary cancer is a brain cancer, grade 1 astrocytoma, grade 2 astrocytoma, grade 3 astrocytoma, glyoblastoma mutiforme, moderate cervical dysplasia (CIN II), early stage cervical cancer, stage IVB cervical cancer, rheumatoid arthritis, multiple sclerosis, systemic lupus, Lyme disease, Dengue fever, hepatitis, amyotrophic lateral sclerosis (ALS), anemia, cardiac disease, diabetes, and renal disease.

20. The method of claims 12, wherein the wherein the samples are selected from: plasma sample, serum sample, a blood sample, an ascites fluid sample, a cerebral spinal fluid sample, a peritoneal fluid sample, a saliva sample, a senovial fluid sample, an ocular fluid sample, and a urine sample.

21. A method of assessing a treatment program for a subject, comprising:
providing a first test sample obtained from the subject at a first time point of interest, occurring before the initiation of the treatment program;
fractionating the first test sample to obtain a first series of fractions;
generating a first series of signature thermograms for the first series of fractions of the first test sample;
providing a second test sample obtained from the subject at a second time point of interest, occurring after the initiation of the treatment program;
fractionating the second test sample to obtain a second series of fractions;
generating a second series of signature thermograms for the second series of fractions of the second test sample;
comparing the first series of signature thermograms to the second series of signature thermograms; and
identifying the treatment program as maintaining the status of the subject when
each second signature thermogram of the fractions of the second sample is a good simulation of the sibling first signature thermograms of the sibling fractions of the first sample; and
identifying the treatment program as changing the status of the subject when at least one second signature thermogram of the fractions of the second sample is a poor simulation of the sibling first signature thermogram of the sibling fraction of the first sample.
